(19) United States
(12) Patent Application Publication
(10) Pub. No.: US 2026/0202556 A1
(43) Pub. Date: Jul. 16, 2026

(54) CODED DETECTION FOR SINGLE PHOTON EMISSION COMPUTED TOMOGRAPHY

(71) Applicant: University of Washington, Seattle, WA (US)

(72) Inventor: Larry A. Pierce, II, Seattle, WA (US)

(73) Assignee: University of Washington, Seattle, WA (US)

(21) Appl. No.: 18/869,763

(22) PCT Filed: May 26, 2023
(86) PCT No.: PCT/US2023/023781

§ 371 Date: Nov. 26, 2024

Related U.S. Application Data
(60) Provisional application No. 63/347,416, filed on May 31, 2022.

(51) Int. Cl. G01T 1/164; A61B 6/03

(52) U.S. Cl. CPC G01T 1/1642; A61B 6/037

(57) ABSTRACT

An example method includes identifying a first number of photons detected by a detector during a first time and when the detector is disposed at a first location and/or first rotation. The example method further includes identifying a second number of photons detected by the detector during a second time and when the detector is disposed at a second location and/or second rotation. In addition, the example method includes determining a value of a pixel or voxel of an image corresponding to a region of a field-of-view (FOV) based on the first number of photons, the first location and/or the first rotation, the second number of photons, and the second location and/or the second rotation.

### CROSS-REFERENCE TO RELATED APPLICATION

This application is a U.S. National Phase patent application based on International Patent Application No. PCT/US2023/023781, filed on May 26, 2023, which claims the priority of U.S. Provisional App. No. 63/347,416, which was filed on May 31, 2022, each of which is hereby incorporated by reference herein in its entirety.

### TECHNICAL FIELD

This application relates to the technical field of medical imaging. In particular, this application describes improvements to Single-Photon Emission Computed Tomography (SPECT), Positron Emission Tomography (PET), and related imaging modalities.

### BACKGROUND

SPECT is a major imaging modality in nuclear medicine. A conventional SPECT imaging system includes a gamma camera configured to detect photons emitted by a radiotracer, which may be injected or otherwise disposed in the body of a patient. The gamma camera is conventionally equipped with a collimator, which restricts the angle at which the photons are received by the gamma camera and prevents photons traveling at different angles from being detected by the gamma camera. A parallel hole collimator, for example, includes one or more parallel holes through which the photons are transmitted from the radiotracer to the gamma camera. Some examples utilize a converging hole collimator, which includes multiple holes extending along directions that converge at a focal point within the body of the patient or outside of the body of the patient. A pinhole collimator includes a small hole through an attenuating plate, wherein the photons from the radiotracer are transmitted through the hole and an image of the radiotracer is projected onto the gamma camera. Due to the presence of a collimator, the photons are received by the gamma camera at known angles. As a result, an image of the radiotracer can be derived based on the total amount of photons received by the gamma camera and the position of the gamma camera.

The collimator of a conventional SPECT imaging system prevents the vast majority of photons emitted by the radiotracer from reaching the gamma camera. The sensitivity of the SPECT imaging system is therefore restricted by the collimator. Due to the reliance on collimators, SPECT conventionally exhibits poorer spatial resolution than positron emission tomography (PET). For instance, clinical whole-body PET scanners may have a 3 mm resolution and SPECT scanners may have a resolution of 10 mm or more. For at least this reason, PET is often preferred over SPECT, particularly for oncological and neurological imaging.

However, SPECT imaging can be performed at a lower cost than PET imaging. Furthermore, there are a greater number of radiotracers that have been determined to be safe and suitable for SPECT imaging than PET imaging, such that SPECT can be used to investigate a greater number of physiological pathways than PET. Further, the positron range and photon acollinearity inherent in PET enforce physical limits on PET resolution.

Common SPECT systems utilized in clinical settings today use essentially the same hardware that was used in the 1950's. Many image reconstruction algorithms adhere to the philosophy that the direction of travel for incoming photons must be known in order to reconstruct an image. Furthermore, many algorithms rely on the use of line-integral methods. It would be advantageous if a SPECT system could be designed with equivalent spatial resolution to a PET system.

### BRIEF DESCRIPTION OF THE DRAWINGS

FIG. 1 illustrates an example environment for performing SPECT imaging using a nonplanar detection surface.

FIGS. 2A to 2C illustrate example environments of a 3-detector array detecting different photon flux from a source 204 at different rotations along an xy plane. FIG. 2A illustrates an environment when the array is in a first rotation. FIG. 2B illustrates an environment when the array is in a second rotation. FIG. 2C illustrates an environment when the array is in a third rotation.

FIGS. 3A to 3C illustrate the environments of FIGS. 2A to 2C at a different perspective.

FIGS. 4A and 4B illustrate an example of a detector, which may have a detection face that is nonplanar to other detection faces of other detectors in an array. FIG. 4A illustrates a cross-sectional view of the detector. FIG. 4B illustrates an example distribution of the flux detected by the detector at a given rotation.

FIG. 5 illustrates an example environment of a detector with different sensitivities to various regions of an FOV.

FIG. 6 illustrates a coded detection (CD) approach to a detector with a planar detection surface.

FIG. 7 illustrates a CD approach to a detector with a nonplanar detection surface.

FIGS. 8A to 8D illustrate cross-sectional views of various detectors and/or detector arrays with nonplanar detection surfaces. FIG. 8A illustrates a detector having a detection surface with rectangular convex and concave portions. FIG. 8B illustrates a detector having a detection surface with triangular convex and concave portions. FIG. 8C illustrates a detector having a detection surface with curved convex portions and rectangular concave portions. FIG. 8D illustrates a detector having an irregular detection surface, with variously shaped convex portions and variously shaped convex portions.

FIG. 9 illustrates a 3D view of a detector with a nonplanar detection surface that is translated and rotated in 3D space.

FIGS. 10A to 10C illustrate an example 3 by 3 detector array in accordance with some implementations. FIG. 10A illustrates a top view of the detector array. FIG. 10B illustrates a cross-sectional view of the detector array. FIG. 10C illustrates another cross-sectional view of the detector array.

FIG. 11 illustrates an example process for performing CD.

FIG. 12 illustrates an example process for fabricating a detector array.

FIG. 13 illustrates an example system configured to perform various methods and functions disclosed herein.

### DETAILED DESCRIPTION

Various implementations described herein relate to improved detectors and image reconstruction techniques for use with medical imaging, such as for SPECT imaging. Some implementations described herein relate to detectors and detector arrays with nonplanar detection surfaces. In various cases, three-dimensional (3D) patterns are etched, embedded, or otherwise disposed in the detection surface. These patterns achieve physical filtering of detection signals that are received at the detection surface, enabling discrimination between the locations of sources of the detection signals. In other words, a topography of the detection surface may alter sensitivities to detection signals (e.g., photons) from various regions in a field-of-view (FOV).

In particular examples, SPECT detectors and detector arrays are described. For instance, the patterns can be present in a detection surface of a single scintillation crystal or an array of scintillation crystals. However, implementations are not so limited. For example, implementations of the present disclosure may apply to ultrasound transducers, PET detection arrays, and other types of detectors. Examples of detection signals include photons, sound, and the like.

In various cases, an image is generated based on a flux of detection signals detected by individual detectors in the imaging system. According to various examples, detectors are translated and rotated through 3D space during image acquisition. As a result of this movement, the sensitivity of individual detectors to detection signals (e.g., photons) emitted from various regions within the FOV can be tuned, optimized, or otherwise changed. Various implementations of the present disclosure utilize these changes in sensitivity to generate images based on the detection signals.

Previous SPECT detector arrays utilized collimators. Collimators, however, present a number of drawbacks. Collimators effectively bock the vast majority of photons emitted by a radiotracer toward a detection array. Thus, the collimator significantly reduces the total number of photons that are detected by the gamma camera. A consequence of low photon count is low image resolution at image reconstruction. Image resolution can be improved by increasing the acquisition time. Thus, a clinically relevant SPECT image may take 10 to 30 minutes to capture. However, such long image acquisition times are inconvenient and uncomfortable for the patient being imaged, who must remain unmoving during image acquisition. Another way to increase image resolution is to increase the radiotracer dose administered to the patient, which increases the number of photons that can be detected by the gamma camera. However, increasing the dose of the radiotracer also increases the dose of radiation received by the patient.

In addition, collimators make traditional SPECT imaging systems unwieldy and unportable, because they have a significant weight. SPECT collimators are typically made of dense materials (e.g., lead) and can weigh hundreds of kilograms (kg), thereby reducing their portability. Further, many SPECT imaging systems are used with different types of collimators for different types of radiotracers, which can take up a significant amount of storage space.

Various implementations of the present disclosure address these and other problems associated with conventional SPECT imaging systems. In examples described herein, an imaging system with a nonplanar detection surface and/or 3D movement and rotation during acquisition can obtain high-resolution and high-sensitivity images of a radiotracer without a collimator. Example imaging systems described herein can be portable. For instance, an imaging system without a collimator may be disposed on a wheeled cart and transported to a patient's bedside in an efficient manner. Thus, various implementations of the present disclosure provide improvements to SPECT image quality and portability.

Presented herein are new hardware configurations for SPECT detector arrays. The disclosed new hardware eliminates the need for collimation of photons in SPECT and can greatly enhance the sensitivity of SPECT detector arrays. The gain in sensitivity can enable in shorter acquisition times and improved image resolution. In some cases, a detector array utilizes high resolution pixelated GAGG crystals coupled to silicon photomultipliers (SiPMs). Elimination of the need for collimation can be accomplished via cutting, etching and grinding patterns into the face of the array in order to separate signals from different positions within the FOV. The array can also be moved with respect to the FOV to further increase signal separability and improve image resolution, though with slightly lowered sensitivity.

A nonplanar, 3D surface of the array can be manufactured by various techniques. For example, an array with some crystals longer than others can be tedious, but relatively simple to assemble. Cutting linear patterns across the face of the crystals would be costly, but not difficult, and could be worth the cost if a pattern is found to have appealing imaging properties (e.g., large singular values in a systems matrix) throughout the FOV. Grinding circular patterns or drilling into the crystal face is possible, but may require great care and could be relatively costly.

By finding a scintillation pattern that yields appealing imaging properties (e.g., large singular values) across the FOV or even in a localized region, it is possible to design a SPECT detector array that has no collimation, thus having a huge sensitivity advantage over collimated systems, and could be tailored to individual purposes (cardiac-focused, brain focused, etc.). This “coded detector” or “CD detector” could be used with other coded detectors to focus on multiple regions in order to have good resolution throughout the FOV, or they could all focus on the same area in order to achieve enhanced local imaging performance at the expense of other parts of the FOV. The coded detector could also be used in conjunction with traditional collimated SPECT detectors in order to add information to the reconstruction algorithm to boost imaging performance, reduce scan time, or reduce radiation dose via the large sensitivity increase.

In order to have more rows than columns in the systems matrix of the CD detector described, the number of scintillation crystal detection elements (rows) must be more than the number of image voxels (columns). This could be challenging, even with 4 detector arrays, unless the crystals are extremely small or if the voxels are very large. In order to expand the number of detector-element rows, the array can be moved in order to further expand the row-space as described herein.

In some implementations, detector motion-induced separability of signals (MISS) is performed. Another way to adjust the flux cosine to scintillation crystals is to use a slant-and-rotate motion of the detector array. Slanting the detector array with respect to the FOV alters the distance from voxels to any given detector. By rotating the slanted detector by 360 degrees, a second degree of freedom is added and this allows for increased separability of signals from individual voxels. For a single panel, the number of rows of a systems matrix can be defined as the number of detectors in the array multiplied by the number of slant angles, multiplied by the number of rotation angles. This can be made to be larger than the number of voxels (columns) even for small voxels. This can allow the systems matrix to have full rank (e.g., no zero singular values).

For a stationary flat-panel array, the signal from a point source can be defined as a 2-dimensional space of (x, z)-values of the position of each crystal in the detector. Step-and-shoot SPECT systems can add a third dimension to this by rotating the array around the FOV, making a richer dataset.

In various cases, reconstruction of a 3D object by line integrals utilized a 4-dimensional parametrized space of lines. SPECT systems that acquire a 3D dataset are generally not performing fully 3D reconstruction, but rather stacks of 2D slices, often with some information-sharing between slices. By stepping the array through many different slant-angles, the flux incident upon a given crystal element from a given voxel is changed and is now represented as a 3D dataset of (x,z) crystal position and slant-angle. Furthermore, the slant also increases or decreases distance to individual voxels, allowing for the possibility of even more separability of signals.

If the detector is also rotated by 360 degrees at each slant angle, the signal space is extended to a fully 4 dimensional (4D) dataset, allowing for greater signal separation (e.g., larger singular values) and thereby improving imaging capabilities.

Both the slant and the rotation can be performed in a step-and-shoot manner. For instance, the detector array motion may be performed in equal or less time than a traditional step-and-shoot collimated system, about 30 minutes. However, since the proposed methods do not use collimation, the count rate to the detector will be increased by a factor of over 2,000. Notably, the slant of the array would cause some loss of sensitivity to the FOV, particularly at greater slant angles. The number of slant and rotate positions would be limited by the photon count rates of individual detectors in the array, enough counts would need to be acquired at each position in order to overcome a variety of the factors that contribute to noise and get a quantitively accurate signal.

Various innovations are described herein, including high-sensitivity, noncollimated photodetector arrays and methods of generating images utilizing these arrays. In some cases, the detection surface of the array is treated to alter flux cosines and improve signal separability for improved imaging performance. In some examples, arrays are subjected to slant-and-rotate movements, which can add additional dimensions that can produce a 4D dataset. According to some implementations, processing can be formed without utilizing line integral-based analysis.

Particular examples will now be described with reference to the accompanying figures.

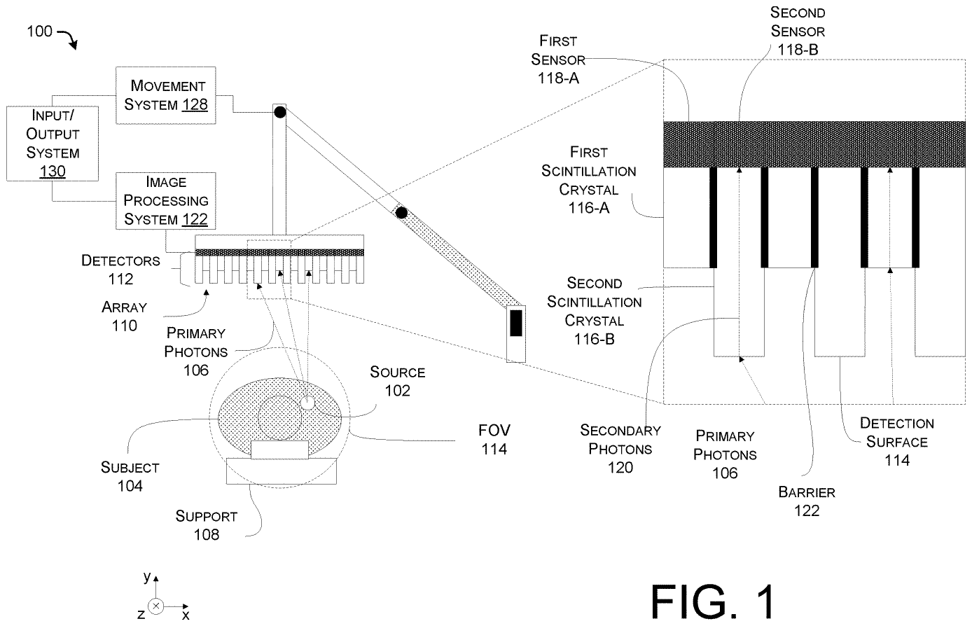

FIG. 1 illustrates an example environment 100 for performing SPECT imaging using a nonplanar detection surface. FIG. 1 illustrates a cross-sectional view of the environment 100 in an xy plane. For reference, a z-direction is perpendicular to the xy plane.

As illustrated in FIG. 1, a source 102 is disposed in a subject 104. In some cases, the subject 104 is a human, such as a patient. In some examples, the source 102 is injected into the subject 104, orally consumed by the subject 104, or otherwise disposed in the subject 104. In particular cases, the source 102 is disposed inside of a physiological structure of the subject 104. As used herein, the term “physiological structure,” and its equivalents, can refer to at least one body part, an organ (e.g., the heart or the brain), one or more blood vessels, or any other portion of a subject. The physiological structure may be associated with a physiological function, which may be an expression of a particular ligand associated with the physiological structure. In some examples, the source 102 is configured to specifically bind to the ligand.

The source 102 is configured to emit primary photons 106. In some cases, the source 102 includes a radiotracer or some other substance configured to emit radiation. For instance, the source 102 may include at least one of technetium-99m, carbon-11, iodine-123, iodine-124, iodine-125, iodine-131, indium-111, copper-64, fluorine-18, thallium-201, rubidium-82, molybdenum-99, lutetium-177, radium-223; astatine-211; yttrium-90; gallium-67, gallium-68, or zirconium-89. In some cases, the source 102 is configured to bind to at least one biomolecule in the subject 104. In various examples, the photons 106 include at least one of x-rays or gamma rays. For instance, at least one of the photons 106 may have an energy of at least 124 electron volts (eV) and less than or equal to 8 MeV, a wavelength of at least 100 femtometers (fm) and less than or equal to 10 nanometers (nm), a frequency of at least 30 petahertz and less than or equal to 10 zettahertz, or any combination thereof. The photons 106 travel through at least a portion of the subject 104. In particular examples, the source 102 is disposed in a brain of the subject 104 and the primary photons 106 travel through a skull of the subject 104.

The subject 104 is disposed on a horizontal or substantially horizontal support 108, such as a bed, stretcher, chair, or other type of padded substrate. In various examples, the primary photons 106 travel through the support 108, such that the support 108 includes a material that is transparent or is otherwise resistant to scattering or absorption of the photons 106. The support 108 is configured to support the subject 104, in various implementations. The subject 104 may be laying down or sitting on the support 108. For example, the support 108 may include a cushioned platform configured to support the weight of the subject 104 during image acquisition.

An array 110 of multiple detectors 112 is configured to detect the primary photons 106 at least partially traversing a volumetric field-of-view (FOV) 114. The primary photons 106 are emitted by the source 102 in the FOV 114. In FIG. 1, the FOV 114 is illustrated as having a circular cross-section in the xy plane. For example, the FOV 114 may be defined as a cylinder. However, implementations are not so limited, and the FOV 114 can be defined as any volumetric shape.

The detectors 112 may be arranged in rows that extend along the z-direction, as illustrated in FIG. 1. Further, although not illustrated in FIG. 1, the detectors 112 in the array 110 may be arranged in columns that extend along the z-direction. Thus, the detectors 112 may be arranged in the array 110 in two dimensions (2D) along an xz plane. In some cases, the row(s) and column(s) of the array 110 extend in directions that are non-perpendicular to one another, such that an angle between the directions is greater than 0 degrees and less than 90 degrees.

A detection surface 114 is defined along the array 110. The detectors 112 are configured to detect the primary photons 106 that cross the detection surface 114. In the example of FIG. 1, each detector 112 may include a scintillation crystal 116 and a sensor 118. For example, a first detector 112 includes a first scintillation crystal 116 coupled to a first sensor 118, and a second detector 112 includes a second scintillation crystal 116 coupled to a second sensor 118. Each scintillation crystal 116 may be a sodium iodide crystal or a GAGG crystal configured to receive a primary photon 106 at the detection surface 114 and to generate a secondary photon 120 based on the received primary photon 106. In various cases, the secondary photon 120 has a lower energy than the primary photon 106. For example, the primary photon 106 may be a gamma ray, whereas the secondary photon 120 may be an optical photon. In various implementations, the number of secondary photons 120 detected by a sensor 118 is substantially equivalent to the number of primary photons 106 received by the scintillation crystal 116 coupled to the sensor 118. Thus, the number of secondary photons 120 detected by a sensor 118 is indicative of the number of primary photons 106 detected by the detector 112. Each sensor 118 may be configured to generate an electrical signal based on a secondary photon 120 generated by its corresponding scintillation crystal 116. For example, each of the sensors 118 may include a semiconductor-based photomultiplier (e.g., a silicon photomultiplier) or another type of photosensor.

Although not illustrated in FIG. 1, in some examples, a detector 112 includes a scintillation crystal 116 that generates an electrical signal in response to receiving a primary photon 106. For instance, the scintillation crystal 116 may include an alloy of cadmium telluride and zinc telluride. Thus, the detector 112, in some examples, may omit a separate sensor 118.

In some implementations, a barrier 122 is disposed between adjacent detectors 112 in the array 110. The barrier 122 may include a material configured to reflect photons, such as BaSO₄; VIKUITI from 3M Corporation of Saint Paul, MN; LUMIRROR from Toray Industries, Inc. of Tokyo, Japan; TiO₂; or any combination thereof.

According to various implementations, each of the detectors 112 is configured to generate a signal (e.g., an electrical signal) based on the detected secondary photons 120 and to provide the signal to an image processing system 122. In some cases, the detectors 112 generate analog signals that are converted to digital signals by one or more analog to digital converters (ADCs) in the image processing system 122. The image processing system 122 is configured to generate the volumetric image of the FOV 114 based on the primary photons 106 that are detected by the detectors 112. In various examples, the image processing system 122 generates an image of the source 102 and/or the subject 104 based on the signals generated by the detectors 112. The image processing system 122 is implemented in hardware and/or software, for instance.

In various cases, the image processing system 122 identifies a flux of the primary photons 106 detected by individual detectors 112 in the array 110, based on the signals output by the sensors in the array 110. As used herein, the terms “flux,” “photon flux,” and their equivalents, can refer to the rate at which photons are received with respect to time. In a discrete environment, a flux of photons can be represented by the number of photons received during a discrete time interval. Flux may also be defined continuously.

According to various examples, the image processing system 122 calculates an image as a collection of voxels respectively representing individual regions of the FOV 114. The number of primary photons 106 detected from an individual region of the FOV 114 may be indicative of a value of the corresponding voxel. In particular cases, the image is a monochromatic image, wherein the value of a given pixel is proportional to the number of primary photons 106 detected from the corresponding region.

In some cases, the image processing system 122 generates the image using the following Equation 1:

$$P f = g \qquad ( 1 )$$

wherein P is a linear operator that describes the physics of the environment 100 (a “systems matrix”), f is an image array, and g is a data array. The image array, for example, is an array including the values of individual voxels in the image. The data array, in various cases, includes fluxes of photons detected by individual detectors 112 in the array 110. Thus, the image can be generated by solving for f.

The systems matrix is dependent on the sensitivities of individual detectors 112 in the array 110 to individual regions within the FOV 114. As used herein, the term “sensitivity,” and its equivalents, may refer to a detector's capability of detecting photons from a given region. For instance, an example sensitivity of an example detector 112 in the array 110 may be a number that is greater than equal to 0 and less than or equal to 1. In various cases, a row of the systems matrix corresponds to a given detector 112 in the array 110. A column of the systems matrix corresponds to a given region in the FOV 114. Thus, a particular row-column element of the systems matrix indicates a sensitivity of a particular detector 112 to a particular region in the FOV 104. The image processing system 122, in various implementations, generates or otherwise identifies the systems matrix by determining the sensitivities of the respective detectors 112 in the array 110.

In theory, in order for the imaging equation to be solvable for the image, the number of rows is at least as many as the number of columns. In other words, theoretically, the number of detectors 112 in the array 110 should be more than the number of regions being imaged in the FOV 114, wherein the regions correspond to voxels in the final image. Further, the quality of the image can be enhanced if singular values (e.g., based on the sensitivities) of the systems matrix are nonzero. Singular values equal to zero in the systems matrix represent a loss of information due to linear dependence of the columns of the imaging matrix, which are also the data vectors for each region of the FOV 114. Thus, singular values equal to zero imply that the image cannot be recovered from the data in the imaging equation, that the null space is non-trivial, and that artifacts or instabilities are likely to be present in the reconstructed image. More zero singular values in the systems matrix means more loss of information and more instability in the reconstruction.

Additionally, singular values close to zero also pose a problem: Equation 1 may be solvable in theory, but small singular values indicate that distinct data vectors are very close to each other in data space, making them indistinguishable in the presence of imaging noise. If signals from two distinct regions in the FOV 114 have very similar data signals detected by the array 110, the imaging system 122 will be unable to resolve the corresponding voxels. Thus, the smaller the singular values in the systems matrix, the more unstable the reconstruction will be and the poorer the resolution of the final computed image. Greater numbers of small singular values in the systems matrix also add to the instability and potential for imaging artifacts. An acceptable lower threshold for singular values generally depends on the problem being solved and the algorithm used to solve it. In order to achieve stable and quantitively accurate image reconstructions, the systems matrix should have minimal singular values that are very small or zero.

A conventional SPECT system, for example, may increase some of the singular values in the imaging matrix by using a collimator. However, in various implementations of the present disclosure, the array 110 and detectors 112 are noncollimated. As used herein, the term “noncollimated,” and its equivalents, may refer to a system that omits or otherwise does not utilize a collimator. As used herein, the term “collimator,” and its equivalents, refers to an object including one or more apertures, wherein the object is configured to attenuate photons that contact the object and to pass other photons transmitted through the aperture(s). Thus, the collimator selectively passes photons that are traveling in paths that extend through the aperture(s). As used herein, an aperture can be an opening in a material specifically designed and created to allow passage of photons approaching from a defined direction. Depending on the narrowness of the aperture(s), the collimator selectively passes photons with substantially predictable directions. For instance, a parallel hole collimator of a conventional SPECT system may selectively pass photons that are within 90±0.5 degrees of a detection surface of a gamma camera. Referring to FIG. 1, a collimator is absent from a space defined between the array 110 and the source 102.

Because the array 110 and/or detectors 112 are noncollimated, the detectors 112 receive a substantial portion of the primary photons 106 emitted from the source 102. This can enhance the number of the primary photons 106 that are received by the detectors 112, because, the primary photons 106 are received at the detectors 112 at a variety of angles. For instance, the first detector 112 receives one or more of the primary photons 106 at an angle that is greater than 0 degrees and less than 85 degrees, 86 degrees, 87 degrees, 88 degrees, 89 degrees, 89.5 degrees, or 89.9 degrees. In some cases, the first detector 112 receives at least two of the primary photons 106, wherein an angle between the paths of the at least two primary photons 106 is between 10 and 170 degrees. For instance, the angle between the primary photons 106 received by the first detector 112 may be 10 degrees, 30 degrees, 40 degrees, 50 degrees, 60 degrees, 70 degrees, 90 degrees, 110 degrees, 130 degrees, 150 degrees, or 170 degrees.

Further, the various drawbacks of collimators described above are not applicable to the system of FIG. 1. However, the lack of collimator presents challenges related to defining the systems matrix. For example, a static, flat detector array without a collimator would result in a systems matrix with small singular values, and therefore would present a challenge for recovering the image array.

According to various implementations of the present disclosure, the systems matrix is enhanced by at least one of two techniques. First, the sensitivities of the detectors 112 to regions within the FOV 114 can be enhanced because the detection surface 114 is nonplanar. Second, the sensitivities of the detectors 112 to regions within the FOV 114 can be enhanced by moving and/or rotating the detectors 112 during a process by which the detectors 112 sense the photons 106. That is, multiple systems matrices can be calculated based on multiple positions and/or rotations of the detectors 112 during image acquisition. Using one or both of these techniques enable implementations of the present disclosure to obtain high-resolution SPECT images of the FOV 114 without the use of a collimator.

In various implementations, which the detection surface 114 is nonplanar, which can increase the singular values represented by the systems matrix. The detection surface 114 of FIG. 1 includes multiple concave portions and multiple convex portions. For instance, the first scintillation crystal 116 extends in a negative y-direction beyond surfaces of its adjacent scintillation crystals among the detectors 112. The second scintillation crystal 116, in contrast, is neighboring scintillation crystals that extend beyond it in the negative y-direction. As a result, the cross-section of the detection surface 114 illustrated in FIG. 1 is nonlinear, and is therefore not defined in a single xz plane. In other words, the detection surface 114 has a 3D pattern.

Because the detection surface 114 is nonplanar, the array 110 physically limits the directionality by which the primary photons 106 are received by individual detectors 112 in the array 110. For instance, the detectors 112 neighboring the second scintillation crystal 116 limit the angle from which the second scintillation crystal 116 can receive the primary photons 106, because they are disposed between at least a portion of the source 102 and the second scintillation crystal 116. Accordingly, the neighboring detectors 112 limit the amount of the FOV 114 from which the source 102 can emit primary photons 106 that are received by the second scintillation crystal 116-B. Furthermore, the neighboring detectors 112 receive a greater number of the primary photons 106 than in implementations in which the array 110 is flat. That is, a portion of the primary photons 106 may be stolen away from one set of the detectors 112 as compared to another set of the detectors 112 in the array 110. In various implementations, concave portions of the detection surface 114 cast shadows on convex portions of the detection surface 114, thereby limiting the angles of primary photons 106 that can be detected by detectors 112 on the convex portions. Some of the detectors 112, therefore, can act as a collimator to at least some other detectors 112 in the array 110.

The distance between the detection surface 114 and the source 102, as well as angle of the detection surface 114 with respect to the primary photons 106, also varies the amount of primary photons 106 detected by the detectors 112. Assuming the source 102 is a point source position (0,y), the photon flux to the point (x,0) on the detection surface 114 is given by Equation 2 (also referred to as “the flux incidence equation”):

$$F ( x , y ) = κ * \frac{1}{\sqrt{{( {x}^{2} + {y}^{2} )}^{2}}} * \frac{y}{\sqrt{{x}^{2} + {y}^{2}}} \qquad ( 2 )$$

Where κ is a proportionality constant. The flux is therefore proportional to the following Equation 3:

$$\frac{1}{\sqrt{{( {x}^{2} + {y}^{2} )}^{2}}} \qquad ( 3 )$$

which is also referred to as “the inverse square law.” According to the inverse square law, the flux to the point (x,0) decreases at a rate proportional to the inverse square of the distance between the point (x,0) and the point source position (0,y) of the source 102. In other words, the photon flux detected by an example detector 112 and the source 102 is proportional to a distance between the detection face of the detector 112 (i.e., the surface at which the example detector 112 receives the primary photons 106) and the source 102 of the primary photons 106. In addition, the flux is proportional to the following Equation 4:

$$\frac{y}{\sqrt{{x}^{2} + {y}^{2}}} \qquad ( 4 )$$

Which is also referred to as the “flux cosine term.” According to Equation 4, the cosine of the angle between a vector normal to the detection face of the example detector 112 is proportional to the flux of the incident primary photons 106 detected by the example detector 112. Although Equations 2 to 4 represent flux and sensitivity in 2D space (e.g., xy space), it is within the scope of one having ordinary skill in the art to adapt Equations 2 to 4 to represent flux and sensitivity in 3D space (e.g., xyz space).

In various implementations, Equation 2 is proportional to a sensitivity of the point (0,y) to the point source at position (x,0). Thus, the systems matrix can be generated based on calculating Equation 2 for individual detectors 112 in the array 110 with respect to various regions of the FOV 114. Accordingly, the sensitivity of a detector 112 to primary photons 106 emitted from a given region in the FOV 114 is dependent on (e.g., proportional to) the distance of that detector 112 to the region in the FOV 114 and the angle of a face of the detector 112 that receives the primary photons 106 with respect to the direction of the primary photons 106.

By altering the shape of the detection surface 114, the incident angle of the primary photons 106 received at the detection surface 114 can be controlled, and as a result, the flux cosine term can be altered in a way that changes the flux of the primary photons 106 detected by the individual detectors 112. Thus, the shape of the detection surface 114 impacts the sensitivity of individual detectors 112 to various regions of the FOV 114. In some cases, the shape of the detection surface 114 can enforce differentiation between fluxes received from neighboring regions of the FOV 114.

In various implementations, as the angle of the primary photons 106 with respect to the detection surface 114 approaches 90 degrees (i.e., as the angle of incidence of the primary photons 106 with respect to the detection surface 114 approaches parallel), the more of the primary photons 106 will be detected by the detectors 112. In contrast, as the angle of the primary photons 106 with respect to the detection surface 114 approaches parallel (e.g., 0 degrees or 180 degrees, or as the angle of incidence approaches 90 degrees), the fewer of the primary photons 106 will be detected by the detectors 112. In an example, the source 102 may be modeled as a point source of the primary photons 106, and a detection face along one of the detectors 112 along the detection surface 114 can be modeled as a square. If the primary photons 106 are transmitted toward the face at a direction normal to the face, then the face may receive (or “catch”) a maximum number of photons 106 at a given distance from the source 102. However, if the face is tilted in any direction, the amount of area that the face can use to receive (or “catch”) the primary photons 106 decreases, thereby resulting in fewer primary photons 106 that are detected by the given detector 112. If the face is entirely parallel with the primary photons 106, then none of the primary photons 106 will be transmitted through the face.

In effect, the topography of the detection surface 114 can act as a physical filter to the photon flux detected by individual detectors 112 in the array 110 in at least two respects. First, convex portions of the detection surface 114 can cast shadows that limit the range of angles at which some detectors 112 receive the primary photons 106, while increasing the number of angles at which the detectors 112 on the convex portions receive the primary photons 106. Thus, the convex portions can act as an alternative to a collimator made of some other material. Second, the topography of the detection surface 114 can tune the sensitivity of the detectors to various regions of the FOV 114 by changing the angles at which the primary photons 106 are received by the detection surface 114.

A variety of shapes can be utilized for the detection surface 114, depending on a desired sensitivity to various regions within the FOV 114 and/or a desired shape of the FOV 114. As shown in FIG. 1, in some implementations, the detectors 112 may individually have flat detection faces, but the detection faces of the detectors 112 may be nonplanar, such that the detection surface 114 is nonplanar.

In some cases, the detection surface 114 may be altered in order to enhance the sensitivity of the detectors 112 to a physiological region of the subject 104, such as a heart, brain, or other organ with particular pertinence. In some cases, the detection surface 114 can be designed to focus the detectors 112 on primary photons 106 emitted from the source 102 in a particular region of the FOV 114. For example, the detection surface 114 can be designed to cause the scintillation crystals 116 to individually or collectively be shaped as a Fresnel lens. In some cases, the detection surface 114 is designed to limit the detectors 112 from detecting the primary photons 106 emitted from the source 102 in another region of the FOV 114. In various implementations, the detection surface 114 includes one or more concave portions, one or more convex portions, one or more ridges, one or more troughs, or any combination thereof. Although FIG. 1 illustrates that each individual scintillation crystal 116 has a rectangular cross-section, implementations are not so limited. In some examples, each individual scintillation crystal 116 may have one or more concave portions, one or more convex portions, one or more ridges, one or more troughs, or any combination thereof. According to some examples, an optically transparent material (e.g., glass, acrylic, epoxy, etc.) may be disposed in one or more concave portions of the detection surface 114. The optically transparent material, for instance, can provide structural support to the array 110 to prevent one or more convex portions of the array 110 from being damaged. The optically transparent material, for instance, is transparent to the primary photons 106.

The systems matrix can be further enhanced by repositioning the array 110 with respect to the FOV 114. In various cases, a movement system 128 is configured to move or otherwise change the position of the detectors 112 in the array 110 with respect to the subject 104 and/or the regions of the FOV 114. According to some implementations, the movement system 128 is configured to translate the array 110 along the x-direction, the y-direction, and the z-direction. Accordingly, the detectors 112 in the array 110 may receive the primary photons 106 at different locations in 3D space. Further, the movement system 128 may be configured to rotate the array 110 along multiple axes. For instance, the movement system 128 may be configured to rotate the array 110 with respect to at least one axis parallel to the x direction, at least one axis parallel to the y direction, and at least one axis parallel to the z direction. The detectors 112 in the array 110 may receive the primary photons 106 at different rotations in 3D space.

The movement system 128, for example, can impact the sensitivity of a given detector 112 to a given region in the FOV 114 in at least three respects. First, by moving the detector 112 closer or farther away from the region in the FOV 114, the movement system 128 can alter the inverse square term represented by Equation 3. Second, by rotating the detector 112, the movement system 128 can alter the flux cosine term represented by Equation 3. Third, by moving and/or rotating the array 110 with the nonplanar detection surface 114, the movement system 128 can selectively cause some detectors 112 to block (i.e., shadow) and unblock other detectors 112 from receiving primary photons 106 from various regions in the FOV 114, which can further impact the sensitivity of the detectors 112 to the regions of the FOV 114. That is, the movement system 128 can alter the shadows cast by at least some of the detectors 112 on other detectors 112 in the array 110. In various implementations, the image processing system 122 recalculates the systems matrix representing the sensitivities of the detectors 112 at each location and/or rotation of the detectors 112 with respect to the regions in the FOV 114.

Various structures in the movement system 128 enable the movement system 128 to reposition (e.g., translate and rotate) the array 110. In various cases, the movement system 128 includes one or more actuators configured to move the array 110. In some examples, the movement system 128 includes one or more arms attached to the array 110 and configured to hold the array 110 in place. According to some examples, the movement system 128 includes one or more motors. In some cases, the movement system 128 further includes one or more sensors configured to identify a current position (e.g., xyz location and/or rotation) of individual detectors 112 in the array 110. The movement system 128, in some examples, includes one or more processors communicatively coupled with the actuators, motors, sensors, or any combination thereof.

In various implementations, the image processing system 122 identifies the photon flux detected by the detectors 112 based on signals output by the detectors 112. The image processing system 122, in various implementations, may generate an image of the FOV 114 including the source 102 based, at least in part, on the shape (e.g., the topography) of the detection surface 114 and the photon flux from the source 102.

According to various cases, the image processing system 122 may calculate or otherwise identify sensitivities of the detectors 112 to individual regions of the FOV 114 based on the shape of the detection surface 114 and/or its orientation with respect to the regions of the FOV 114. The sensitivity of a detector 112 at a particular location and/or rotation to a region in the FOV 114 may be impacted by whether the detector 112 is shadowed from the region, an angle of a detection face of the detector 112 along the detection surface 114 with respect to a direction between the region of the FOV 114 and the detector 112, as well as a distance between the detector 114 and the region of the FOV 114. For example, a detector 112 that is blocked (e.g., by another detector 112) from receiving primary photons 106 from a region of the FOV 114 may have a sensitivity to that region that is equal to 0. In contrast, a detector 112 that is at least partially exposed to the region may have a sensitivity to that region that is greater than 0. In various cases, a detector 112 that is greater than a threshold distance from a region of the FOV 114 may have a sensitivity to that region that is less than a threshold sensitivity. Relatedly, a detector 112 that is less than the threshold distance from the region of the FOV 114 may have a sensitivity to that region that is greater than the threshold sensitivity. According to various examples, a detector 112 whose detection face is normal with respect to a line extending from a region of the FOV 114 to the detector 112 (also referred to as a “line-of-response” or LOR) may have a sensitivity to that region that is greater than a threshold sensitivity. In contrast, a detector 114 whose detection face is parallel with respect to the LOR extending from the region of the FOV 114 may have a sensitivity to that region that is equal to or approaching 0. Relatedly, a detector 112 whose detection face is tilted with respect to the ray, but is not parallel to the LOR, may have a nonzero sensitivity to that region.

In various cases, the imaging processing system 122 identifies the location and/or rotation of the array 110 based on one or more signals from the movement system 128. In some examples, the movement system 128 further indicates the time, velocity, acceleration, or any combination thereof, of the array 110. The image processing system 122 may generate the image of the source 102 based on the location and/or rotation of the array 110. For example, the image processing system 122 may identify sensitivities to individual regions of the FOV 114 based on the topography of the detection surface 114, the location of the detectors 112 within 3D space, as well as the rotation of the detectors 112 with respect to the regions of the FOV 114. The image processing system 122 may generate the systems matrix (e.g., may calculate the singular values of the systems matrix) based on the sensitivities of the detectors 112 to the regions of the FOV 114. The image processing system 122 may generate an image of the FOV 114 and/or the source 102 based on the systems matrix and the flux and/or photon counts reported by the detectors 112.

The environment 100 further includes an input/output system 130 that is communicatively coupled to the image processing system 122 and the movement system 128. The input/output system 130 is configured to receive input signals from a user, such as a clinician or imaging technician. In various cases, input signals indicate an instruction to begin detecting the primary photons 106 and/or to generate the image of the FOV 114. In response, the input/output system 130 may output signals to the image processing system 122 and/or the movement system 128 to begin obtaining the image. In various implementations, the input signals may indicate a particular region of the FOV 114 to be imaged. For example, a clinician may input an indication that a heart of the source 104 is of particular clinical interest. Various input signals may be received via any of possible input devices, such as keyboards, touchscreens, microphones, and the like.

The input/output system 130 may output signals to the image processing system 122 and/or the movement system 128 to position the array 110 in order to optimize receipt and/or differentiation of primary photons 106 from the particular region of the FOV 114. In various implementations, the input/output system 130 may further output the image generated by the image processing system 122. For example, the input/output system 130 may include a transceiver configured to transmit a signal indicative of the image to an external device. In some cases, the input/output system 130 may include a display (e.g., a screen, a holographic display, etc.) configured to visually present the image. The input/output system 130 may be implemented in hardware and/or software.

In particular implementations, the environment 100 is utilized to image the subject 104. For example, the source 102 may be a radiotracer conjugated to an antibody that specifically binds a target expressed by a tumor of the subject 104. After receiving the source 102, the subject 104 may be asked to lay down on the support 108. In conventional SPECT systems, the support 108 may be located in a room that is specifically designed for SPECT imaging, because a conventional SPECT system may utilize a heavy and large collimator to detect the primary photons 106 from the source 102. In contrast, according to some implementations, the array 110 may detect the primary photons 106 emitted from the source 102 without a collimator. Therefore, the support 108 could be located in a variety of settings within a hospital, such as in a medical ward environment, procedure room, or general examination room. Further, the array 110, the image processing system 122, the movement system 128, the input/output system 130, or any combination thereof, may be disposed on a wheeled cart (not illustrated) that can be transported to the location of the subject 104 by a single care provider (e.g., a nurse, a medical technician, a physician, or the like).

During image acquisition, the movement system 128 may move the array 110 to different locations and/or rotations around the FOV 114. The image processing system 122, for instance, may identify the photon flux detected by each individual detector 112 at each location and/or rotation. Further, the image processing system 112 may identify the systems matrix of the detectors 112 at each location and/or rotation. Based on the photon fluxes and systems matrices, the image processing system 112 may generate an image of the FOV 114 that indicates the location of the source 102 of the primary photons 106. The input/output system 130, for instance, may display the image to the care provider. In turn, the care provider may study the image in order to identify a location and type of the tumor of the subject 104. Accordingly, the care provider may facilitate a targeted treatment (e.g., a surgery, an oncologic treatment, etc.) of the tumor based on the image.

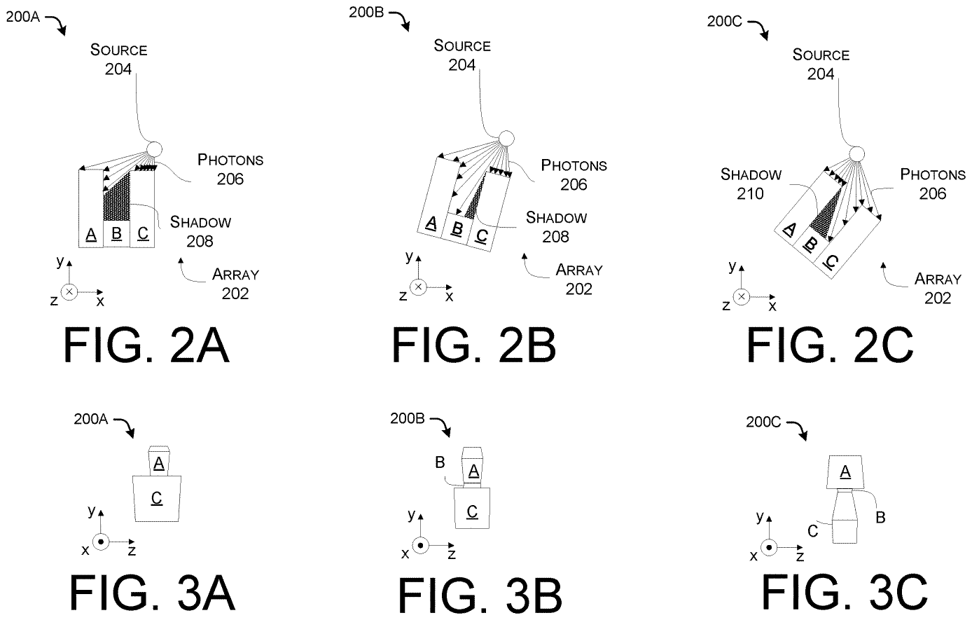

FIGS. 2A to 2C illustrate example environments 200A, 200B, and 200C of a 3-detector array 202 detecting different photon flux from a source 204 at different rotations along an xy plane. The rotations illustrated in FIGS. 2A to 2C cause the fluxes to change based on detector shadowing, distance, and angle with respect to the source 204. FIG. 2A illustrates an environment 200A when the array 202 is in a first rotation. FIG. 2B illustrates an environment 200B when the array 202 is in a second rotation. FIG. 2C illustrates an environment 200C when the array 202 is in a third rotation. The array 202 includes a detector A, a detector B, and a detector C. In various examples, the array 202 could be at least a portion of the array 110 described above with respect to FIG. 1. In some examples, the source 204 is the source 102 described above with reference to FIG. 1. In some implementations, the detectors A, B, and C correspond to the detectors 112 described above with reference to FIG. 1.

The source 204 is configured to emit photons 206 radially along the xy plane. In various cases, the photon flux of the photons 206 on an area decreases as the area is farther from the source 204, due to Equation 3. In various implementations, the photon flux of the photons 206 is also dependent on an angle of the area with respect to a plane normal to the photons 206, due to Equation 4. The detectors A, B, and C, have different distances from the source 204, and different angles with respect to the photons 206 emitted from the source 204, at the first rotation, the second rotation, and the third rotation. Accordingly, the photon flux detected by the detectors A, B, and C is different at the first rotation, the second rotation, and the third rotation.

In FIG. 2A, the detector C casts a shadow 208 over detector B and partially over detector A, such that detector B is unable to receive any of the photons 206 from the source. Detector A and detector C both receive photons 206 in the first rotation. However, because the upper face of detector C is locate closer to the source 204 and is more normal to the photons 206 it receives, the flux detected from the upper face of detector C is greater than the upper face of detector A. That said, detector A also has a side face exposed to the source 204, which increases the number of the photons 206 that detector A receives. In the first rotation, detector A detects a photon flux that is proportional to four (indicated by the four photons 206 illustrated as being transmitted onto detector A), detector B detects a photon flux that is proportional to zero, and detector C detects a photon flux that is proportional to six.

In FIG. 2B, the detector B is only partially obscured by the shadow 208 that is cast by detector C. Thus, each one of the detectors A, B, and C receive photons 206 in the second rotation. In the second rotation, the detector A receives more of the photons 206 than in the first rotation, due at least partially to the shadow 208 moving away from the side face of detector A. Detector B receives at least one photon 206, but due to its partially occluded detection face and distance from the source 204, detector B detects fewer photons 206 than either detector A or detector C. Finally, detector C detects fewer of the photons 206 than at the first rotation, due to the increased distance between the source 204 and the detection face of detector C. In the second rotation, detector A detects a photon flux that is proportional to five, detector B detects a photon flux that is proportional to one, and detector C detects a photon flux that is proportional to five.

In FIG. 2C, the detector A casts a shadow 210 partially over detector B and over the side face of detector A, such that detector A only receives the photons 206 from its top face. Due to the reduced distance between the top face of detector A and the source 204, as well as the fact that the top face is substantially normal to the photons 206 it receives, the top face of detector A receives a substantial number of photons at the third rotation. Due to its distance from the source 204 and the shadow 210, detector B detects minimal photons 206. Detector C finally has a side face exposed to the source 204 in the fourth rotation, which increases the total surface area by which detector C receives the photons 206. However, detector C is farther from the source 204 in the third rotation than at the first or second rotations. In the third rotation, detector A detects a photon flux that is proportional to five, detector B detects a photon flux that is proportional to one, and detector C detects a photon flux that is proportional to six.

Due to the different photon fluxes detected by the detectors A, B, and C at the different rotations, and knowledge about the overall shape of the detection surface of the array 202, an imaging system may determine the location of the source 204.

FIGS. 3A to 3C illustrate the environments 200A, 200B, and 200C of FIGS. 2A to 2C at a different perspective. Namely, FIGS. 3A to 3C illustrate the environments 200A, 200B, and 200C from the perspective of the source 204. The photon fluxes of the respective detectors A, B, and C at each rotation are related to the area at which the source 204 projects the photons 206 onto the detectors A, B, and C. These areas are illustrated in FIGS. 3A to 3C.

The areas of the detectors A, B, and C presented in FIGS. 3A to 3C are proportional to the photon fluxes described with respect to FIGS. 2A to 2C. For example, at the first rotation illustrated in FIG. 3A, the areas in order of largest to smallest are C>A>B=0. At the second rotation illustrated in FIG. 3B, the areas in order of largest to smallest are C=A>B>0. At the third rotation illustrated in FIG. 3C, the areas in order of largest to smallest are C>A>B>0.

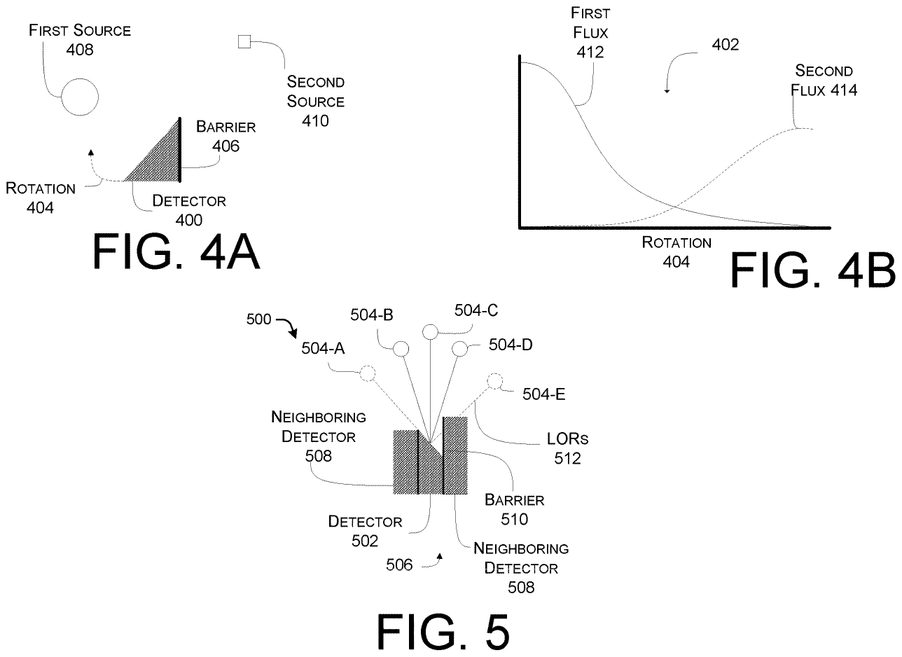

FIGS. 4A and 4B illustrate an example of a detector 400, which may have a detection face that is nonplanar to other detection faces of other detectors in an array. FIG. 4A illustrates a cross-sectional view of the detector 400. FIG. 4B illustrates an example distribution 402 of the flux detected by the detector 400 at a given rotation 404.

The detector 400 illustrated in FIG. 4 has a detection surface that includes a detection face 404. The detector 400 has a convex ridge that extends in a y direction. In addition, the detector 400 includes a barrier 406 disposed on another face of the convex ridge that is parallel with the y-direction at an initial rotation angle.

FIG. 4A also illustrates a first source 408 and a second source 410. At the initial rotation angle (e.g., a rotation of 0 degrees), the detector 400 is configured to receive a substantial amount of photons from the first source 408, because the face 404 is substantially normal to a direction between the detector 400 and the first source 408. At the initial rotation angle, the detector 400 is unable to detect photons from the second source 410, because the direction of a ray extending between the detector 400 and the second source 410 is substantially parallel to the face 404. Notably, the first source 408 is closer to the detector 400 than the second source 410. The detector 400 detects a first flux 412 from the first source 408 and a second flux 414 from the second source 410.

The first flux 412 and the second flux 414 both change as the detector 400 is rotated. Namely, the first flux 412 decreases as the detector 400 is rotated from 0 degrees to 90 degrees. However, the second flux 414 increases as the detector 400 is rotated from 0 degrees to 90 degrees. The first flux 412 is dependent on the changing sensitivity of the detector 400 to a region including the first source 408 as the detector 400 rotates. Further, the second flux 414 is dependent on the changing sensitivity of the detector 400 to a region including the second source 410 as the detector 400 rotates.

In various implementations, the detector 400 detects photons corresponding to a combination of the first flux 412 and the second flux 414. However, the first flux 412 and the second flux 414 may be distinguished from another based on fluxes detected by other detectors in an array. Furthermore, the peak caused by the first source 408 may be larger than the peak of the second source 410, due to the shorter distance between the detector 400 and the first source 408 as compared to the distance between the detector 400 and the second source 410. In effect, the total photon flux distribution with respect to angle may have two local maxima-one corresponding to the peak of the first source 408 and one corresponding to the peak of the second source 410. The imaging system may identify the locations of the first source 408 and the second source 410 based on the local maxima in the photon flux distribution.

In various cases, the detector 400 can be modified to further change the photon flux distribution, and to potentially result in further separation of the local maxima of the photon flux distribution with respect to rotation angle. In some examples, the detector 400 may be neighbored by additional detectors that, collectively with the detector 400, result in a nonplanar detection surface. The nonplanar detection surface can change the photon flux distribution due to shadowing and/or detection angle. Further, the detector 400 may be translated around xy space, or may be rotated along a different rotational axis that is nonparallel to the z direction. These changes may further produce features within the photon flux distribution that enable the first source 408 to be differentiated from the second source 410 within the FOV.

FIG. 5 illustrates an example environment 500 of a detector 502 with different sensitivities to various regions 504-A to 504-E of an FOV. The detector 502 is part of an array 506 that includes neighboring detectors 508. A barrier 510 is disposed between the detector 502 and the neighboring detectors 508 in the array 506. As shown, the array 506 has a nonplanar detection surface. That is, a detection face of the detector 502 is nonparallel to a detection face of each of the neighboring detectors 508.

The detector 502 may have a first sensitivity to a first region 504-A. The first sensitivity is equal to zero, because an LOR 512 extending from the first region 504-A to the detector 502 is parallel to the detection face of the detector 502.

The detector 502 has a second sensitivity to a second region 504-B, a third sensitivity to a third region 504-C, and a fourth sensitivity to a fourth region 504-D. The second, third, and fourth sensitivities are each nonzero. As shown in FIG. 5, LORs 512 from the second region 504-B, the third region 504-C, and the fourth region 504-D extend to the detection face of the detector 502 at nonparallel angles. Further, the LORs 512 from the second region 504-B, the third region 504-C, and the fourth region 504-D are not intersected or otherwise blocked by the neighboring detectors 508. In various implementations, the sensitivity of the detector 502 to a given LOR 512 is dependent on the angle between the LOR 512 and the detection face of the detector 502. In various implementations, the fourth sensitivity may be higher than the third sensitivity, which may be higher than the second sensitivity. This is at least in part due to the angle between the LOR 512 from the fourth region 504-D being closest to normal to the detection face, and the angle between the LOR 512 from the second region 504-B being closest to parallel to the detection face.

In addition, the detector 502 may have a fifth sensitivity to a fifth region 504-E. Because an LOR 512 extending from the fifth region 504-E to the detection face of the detector 502 is intersected by the neighboring detector 508, the fifth sensitivity may be equal to zero.

In various implementations, an image depicting a source of photons disposed in an FOV including the first to fifth regions 504-A to 504-E can be generated based on a photon flux detected by the detector 502 and the first to fifth sensitivities. Further, the array 506 may be repositioned and the image may be calculated further based on the resultant sensitivities and the photon flux detected at that time. In various implementations, the image may be defined as an array of pixels or voxels that respectively depict the first to fifth regions 504-A to 504-E in the FOV.

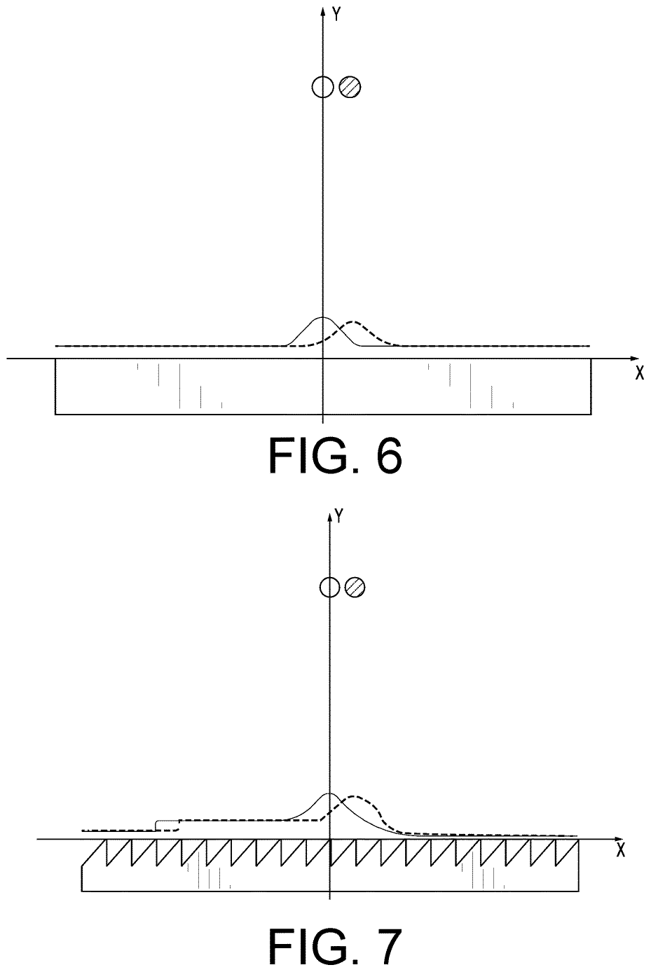

FIG. 6 illustrates a coded detection (CD) approach to a detector with a planar detection surface. For example, FIG. 6 may depict an example of a flat scintillation crystal. For a point source at position (0,y), the photon flux to the point (x,0) on the detector face is given by the flux incidence equation (Equation 2).

Notably, even a modest y-value makes the flux nearly constant in x, meaning that the data vectors from neighboring voxels will be nearly identical, or essentially linearly dependent. This is equivalent to the systems matrix having small singular values, and can lead to poor reconstruction resolution and possible artifacts or instabilities. In the flat detector model, the value of Equation 3 can be altered by changing the distance from the source to the detector. Further, the value of Equation 4 can be altered by changing the rotation angle of the detector with respect to the source. Thus, in some cases, even a flat, noncollimated detector can be used to identify volumetric information within a FOV if the flat detector is translated and rotated in various directions and rotation axes.

FIG. 7 illustrates a CD approach to a detector with a nonplanar detection surface. In various implementations, Equation 4 can be altered by changing the shape of the detection surface of the detector. In various cases, the shape of the detection surface can create rapid changes in signals with respect to sources in different regions of the FOV, as the detector is moved in 3D space, and as the detector is rotated around multiple axes. The nonplanar detection surface can create distinguishing features in the flux signal to allow for greater separability of signals from nearby regions in the FOV, which is equivalent enlarging the singular values of the systems matrix for some local subset of FOV regions or for the entire FOV.

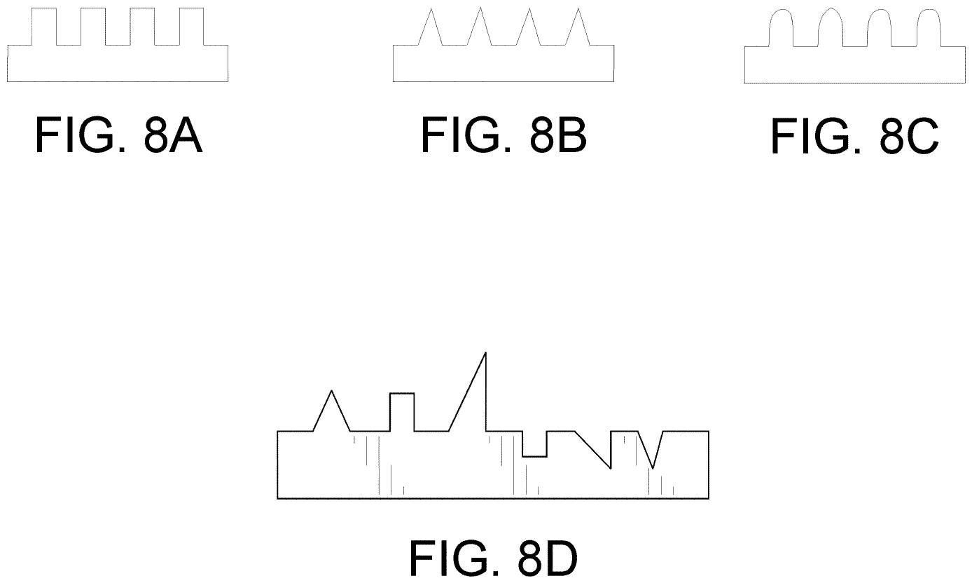

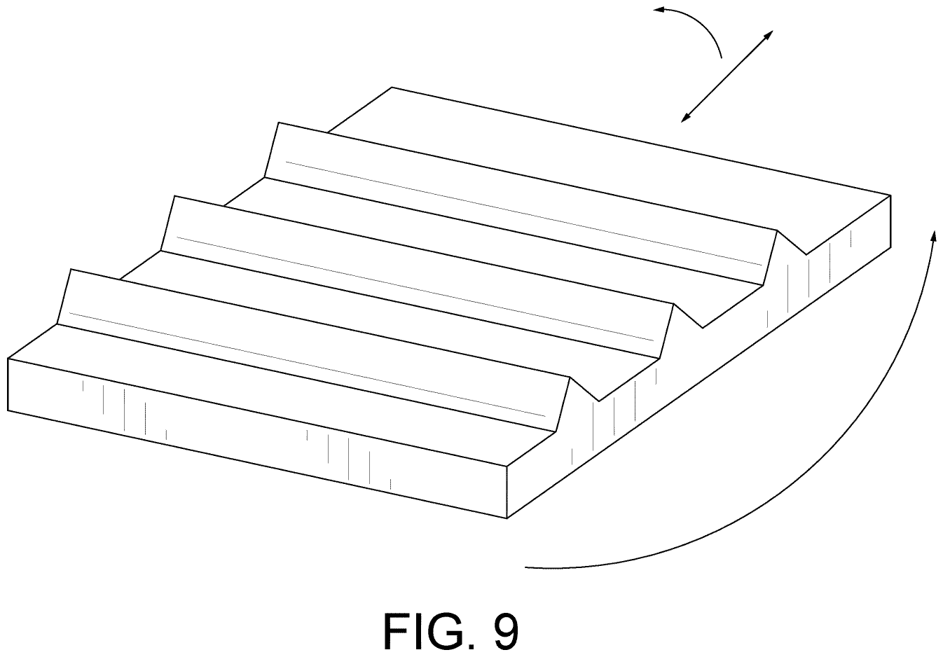

FIGS. 8A to 8D illustrate cross-sectional views of various detectors and/or detector arrays with nonplanar detection surfaces. FIG. 8A illustrates a detector having a detection surface with rectangular convex and concave portions. FIG. 8B illustrates a detector having a detection surface with triangular convex and concave portions. FIG. 8C illustrates a detector having a detection surface with curved convex portions and rectangular concave portions. FIG. 8D illustrates a detector having an irregular detection surface, with variously shaped convex portions and variously shaped convex portions.

Creating signal-distinguishing patterns can be accomplished by having some detectors (e.g., scintillation crystals) be taller than others cutting or etching linear patterns into the face, or by having circular patterns ground or drilled into the detection faces of the detectors. Other methods are also possible. Having different patterns in the detection face will lead to different outcomes, with some patterns having increasing resolution in the x-direction, and others increasing resolution in the y-direction. Some patterns may have excellent imaging properties localized to a small region with poor imaging outside that region.

There are many possibilities of patterns to be used on the face of the detector, and inspiration can be taken from Fresnel lenses, zone plates, coded apertures, wavelets, and even the Fourier Transform to design patterns on the scintillation crystal face. As shown in FIGS. 8A to 8D, detectors may have different patterns of different heights, linear cuts, and circular grindings as well as different cross-sectional profiles.

FIG. 9 illustrates a 3D view of a detector with a nonplanar detection surface that is translated and rotated in 3D space. In various implementations, the nonplanar detection surface of the detector can alter the photon flux detected from one or more sources in a 3D FOV. The translation and rotation of the detector can further alter the photon flux. Accordingly, an imaging system may generate a high-quality image of the one or more sources using the photon flux detected by the detector.

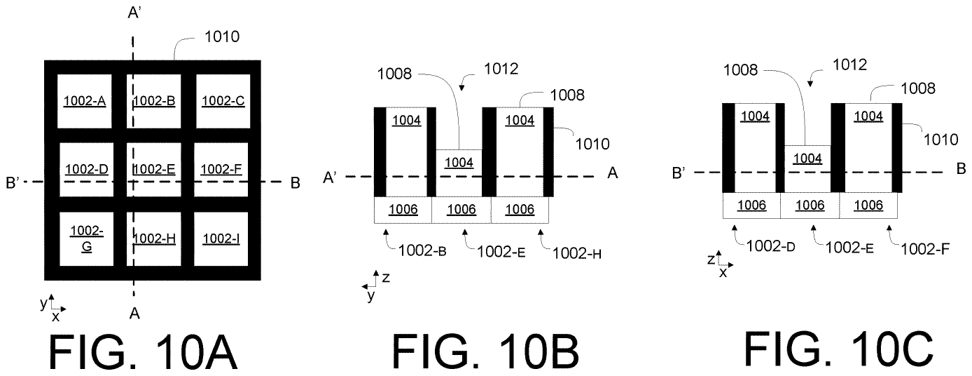

FIGS. 10A to 10C illustrate an example 3 by 3 detector array 1000 in accordance with some implementations. FIG. 10A illustrates a top view of the detector array 1000. FIG. 10B illustrates a cross-sectional view of the detector array 1000. FIG. 10C illustrates another cross-sectional view of the detector array 1000. The line A′ is illustrated in both FIGS. 10A and 10B. The line B′ is illustrated in both FIGS. 10A and 10C. In various implementations, the detector array 1000 includes first through ninth detectors 1002-A to 1002-1. The detectors 1002-A to 1002-I are arranged in three rows extending in an x direction and three columns extending in a y direction.

Each of the detectors 1002-A to 1002-1 includes a crystal 1004 and a sensor 1006. The crystal 1006 includes a detection face 1008, at which photons are received. The crystal 1004 may be configured to generate relatively low-energy photons (e.g., visible light) based on receiving relatively high-energy photons (e.g., x-rays or gamma rays) from the FOV of the detector array 1000. The low-energy photons may be sensed by the corresponding sensor 1006.

To avoid the relatively low-energy photons from traveling between the crystals 1004, a barrier 1010 may be disposed between the crystals 1004. The barrier 1010 may include a material configured to reflect the relatively low-energy photons. Accordingly, the low-energy photons received by the sensor 1006 of a particular detector 1002 may correspond to a high-energy photon received by the crystal 1004 of the particular detector 1002.

Further, the detection faces of the respective detectors 1002-A to 1002-I may collectively embody a detection surface 1012 of the array 1000. The detection surface 1012, in various implementations, is nonplanar. For instance, a detection face 1008 of the detector 1002-E may be nonplanar with respect to a detection face 1008 of the detector 1002-H.

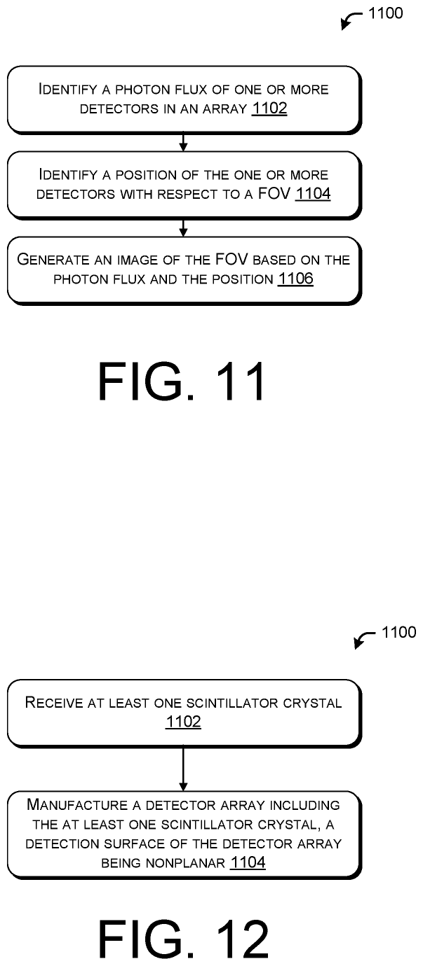

FIG. 11 illustrates an example process 1100 for performing CD. The process 1100 is performed by an entity, such as one or more processors, a computing device, an imaging system (e.g., the image processing system 122), or any combination thereof.

At 1102, the entity identifies a photon flux of one or more detectors in an array. In some implementations, the array has a nonplanar detection surface. For instance, an example detector has a nonplanar detection face. In some cases the array is moved between different locations and/or rotations, and the photon flux is detected at the different locations and/or rotations. According to various cases, the photon flux detected by a particular detector is calculated based on the number of photons detected by the particular detector during a particular time interval.

At 1104, the entity identifies a position of the one or more detectors with respect to an FOV. In some cases, the position represents the location and/or rotation of the detector(s) at the different locations and/or rotations. In various implementations, the position represents a topography of the detection surface with respect to various regions in the FOV. In some implementations, the entity generates or otherwise identifies a systems matrix representing at least one sensitivity of the one or more detectors to various regions in the FOV. For instance, the entity may determine the sensitivity of an example detector to an example region in the FOV based on Equation 2. In some cases, the sensitivity of a given detector to a given region in the FOV is calculated based on one or more LORs extending from the given region to the given detector. The sensitivity is based on, for example, a length of the LOR(s) (e.g., a distance between the given region and the given detector, as provided by Equation 3), an angle of the LOR(s) with respect to a detection face of the given detector (e.g., as provided by Equation 4), and whether any of the LOR(s) are shadowed or blocked (e.g., intersected by another detector). In various implementations, the given detector has different sensitivities to the given region of the FOV at different locations and/or rotations. According to some cases, the sensitivity of the given detector to the given region is dependent on the topography of the detection surface.

At 1106, the entity generates an image of the FOV based on the photon flux and the position. In various implementations, the entity solves Equation 1 in order to obtain an image array including values of pixels or voxels that represent respective regions in the FOV. In some implementations, the entity generates the image of the FOV by calculating a derivative of the photon flux over time, wherein the detector(s) are configured to detect the photons while moving through 3D space between different locations and/or rotations.

FIG. 12 illustrates an example process 1200 for fabricating a detector array. The process 1200 may be performed by an entity including, for instance, a user, a machine, a computing device, at least one processor, or any combination thereof.

At 1202, the entity receives at least one scintillator crystal. According to various implementations, the scintillator crystal(s) includes at least one of cerium-doped multicomponent gadolinium aluminum gallium garnet (Ce: GAGG) or an alloy of cadmium telluride and zinc telluride.

At 1204, the entity manufactures the detector array including the at least one scintillator crystal. A detection surface of the detector array is nonplanar. In some cases, the entity grinds or cuts patterns into individual scintillator crystals. A detection face of an example scintillator crystal, for instance, is manufactured to include one or more convex portions and/or one or more concave portions. In some cases, the entity places multiple scintillator crystals into an array, wherein the collective detection surface of the array is nonplanar. For instance, the detection faces of the scintillator crystals can be disposed at multiple levels, multiple angles, or the like. In some cases, the entity couples one or more sensors (e.g., photosensors) to the scintillator crystal(s).

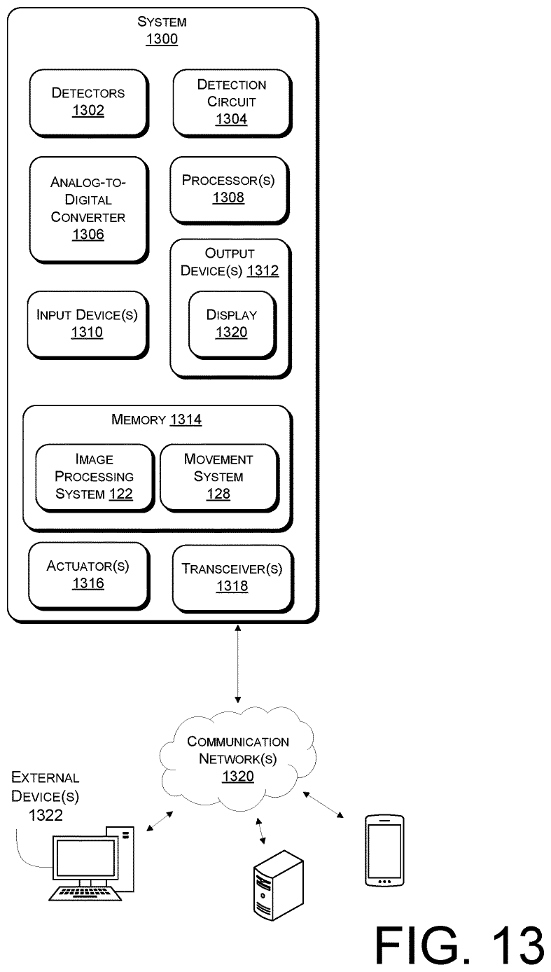

FIG. 13 illustrates an example system 1300 configured to perform various methods and functions disclosed herein. The system 1300 includes detectors 1302, a detection circuit 1304, an analog-to-digital converter 1306, one or more processors 1308, one or more input devices 1310, one or more output devices 1312, memory 1314, one or more actuators 1316, and one or more transceivers 1318. In some implementations, any of these components may be omitted from the system 1300.

The detectors 1302 may be configured to receive photons from an FOV of the system 1300. The photons, for example, may be x-rays, gamma rays, or a combination thereof. In various implementations, the detectors 1302 may be configured to generate analog signals based on the photons they receive from the FOV.

The detection circuit 1304 may be an electrical circuit configured to receive the analog signals generated by the detectors 1302. In various examples, the detection circuit 1304 may include one or more analog filters configured to filter the analog signals. In some cases, the detection circuit 1304 includes a thresholding circuit configured to filter out analog signals generated based on photons received by the detectors 1302 at energy levels below a threshold energy level. Accordingly, the system 1300 may ignore photons from the FOV that have been scattered before reaching the detectors 1302.

The analog-to-digital converter 1306 may convert the analog signals from the detection circuit 1304 into one or more digital signals. The analog-to-digital converter may provide the digital signal(s) to the processor(s) 1308 for further processing. The digital signal(s) may be indicative of the fluxes of photons detected by the detectors 1302 over time.

In some implementations, the processor(s) 1308 include a central processing unit (CPU), a graphics processing unit (GPU), or both CPU and GPU, or other processing unit or component known in the art. The processor(s) 1308 may be configured to execute instructions stored in the memory 1314, in various implementations. In some examples, the processor(s) 1308 are configured to generate an image of the FOV based on the digital signal(s) generated by the analog-to-digital converter 1306.

The input device(s) 1310 may include, for instance, a keypad, a cursor control, a touch-sensitive display, voice input device, etc. In some implementations, the input device(s) 1310 are configured to receive an input signal (e.g., from a user) requesting a relatively high-resolution image of a portion of the FOV. The input device(s) 1310 may be communicatively coupled to the processor(s) 1308 and may indicate the input signal to the processor(s) 1308. The output device(s) 1312 may include, for example, a display 1320, speakers, printers, etc. The output device(s) 1312 may be communicatively coupled to the processor(s) 1308. In various implementations, the display may be configured to output the image of the FOV generated by the processor(s) 1308.

The memory 1314 may include various instruction(s), program(s), database(s), software, operating system(s), etc. In some implementations, the memory 1314 includes instructions that are executed by processor(s) 1308 and/or other components of the system 1300. For example, the memory 1314 may include software for executing functions of the image processing system 130 and/or movement system 138 described above with reference to FIG. 1. For example, the processor(s) 1308, upon executing instructions of the image processing system 130, may be configured to generate an image of the FOV based on the digital signal(s) generated by the analog-to-digital converter 1306. In some cases, the processor(s) 1308 may further generate the image based on one or more signals from the actuator(s) 1316, which may be indicative of positions of the detectors 1302. According to some examples, the instructions in the movement system 138, when executed by the processor(s) 1308, may cause the processor(s) 1308 to perform operations including controlling the actuator(s) 1316 to move the detectors 1302 (e.g., at a particular speed, in a particular position, etc.).

The device system 1300 include additional data storage devices (removable and/or non-removable) such as, for example, magnetic disks, optical disks, or tape. Tangible computer-readable media can include volatile and non-volatile, removable and non-removable media implemented in any method or technology for storage of information, such as computer readable instructions, data structures, program modules, or other data. The memory 1314, the removable storage, and the non-removable storage are all examples of computer-readable storage media. Computer-readable storage media include, but are not limited to, Random Access Memory (RAM), Read-Only Memory (ROM), Electrically Erasable Programmable Read-Only Memory (EEPROM), flash memory, or other memory technology, Compact Disk Read-Only Memory (CD-ROM), Digital Versatile Discs (DVDs), Content-Addressable Memory (CAM), or other optical storage, magnetic cassettes, magnetic tape, magnetic disk storage or other magnetic storage devices, or any other medium which can be used to store the desired information and which can be accessed by the system 1300. Any such tangible computer-readable media can be part of the system 1300. In some examples, the processor(s) 1308 may be configured to perform various functions described herein based on instructions stored on a non-transitory computer readable medium.

In various implementations, the actuator(s) 1316 may include one or more motors configured to move and/or rotate the detectors 1102. The actuator(s) 1316 may be communicatively coupled with the processor(s) 1308.

The system 1300 can be configured to communicate over a telecommunications network using any common wireless and/or wired network access technology. For example, the transceiver(s) 1318 can include a network interface card (NIC), a network adapter, a Local Area Network (LAN) adapter, or a physical, virtual, or logical address to connect to various network components, for example. To increase throughput when exchanging wireless data, the transceiver(s) 1318 can utilize multiple-input/multiple-output (MIMO) technology. The transceiver(s) 1318 can comprise any sort of wireless transceivers capable of engaging in wireless, radio frequency (RF) communication. The transceiver(s) 1318 can also include other wireless modems, such as a modem for engaging in Wi-Fi, WiMAX, Bluetooth, infrared communication, and the like. The transceiver(s) 1318 may include transmitter(s), receiver(s), or both. In various implementations, the transceiver(s) 1318 can transmit data over one or more communication networks 1320, such as at least one Wi-Fi network, at least one WiMAX network, at least one Bluetooth network, at least one cellular network, one or more wide area networks (WANs), such as the Internet, or the like. The transceiver(s) 1318 may transmit the data to one or more external devices 1322, such as external computing devices. The transceiver(s) 1318 may be communicatively coupled to the processor(s) 1308. For example, the processor(s) 1308 may generate data indicative of the image of the FOV, and the transceiver(s) 1318 may transmit that data to the external device(s) 1322.

The system 1300 may be configured to communicate over the communications network(s) 1320 using any common wireless and/or wired network access technology. Moreover, the system 1300 may be configured to run any compatible device Operating System (OS), including but not limited to, Microsoft Windows Mobile, Google Android, Apple iOS, Linux Mobile, as well as any other common mobile device OS.

Although various implementations are described herein with reference to SPECT and PET tomography, it will be obvious to persons of skill in the art, based on the present disclosure, that the disclosed systems may be used to perform tomosynthesis (e.g., high resolution limited-angle tomography), other planar imaging, or non-tomographic imaging as is known in the art. Any method that utilizes an attenuating object that is systematically moved during image acquisition so as to alter detector flux and thus enables the creation of an imaging dataset or enables the computation of flux or count rates from specific lines of response is contemplated.

Various noncollimated imaging systems described herein can be used for PET imaging. In PET, two anti-parallel photons are detected in a pair and the line of response used in image reconstruction is determined by the positions of the two photon interactions. If a collimator is removed from a detector, pairs of the fully exposed detectors can act as PET detectors. The photons used in PET can be 511 keV each, and are generally much higher energy than the photons used in SPECT imaging. If both a PET and SPECT tracer are in the field of view at the same time, discriminating between the PET and SPECT photon energies could allow for the simultaneous acquisition of both SPECT and PET data.

### EXAMPLE CLAUSES

1. A single photon emission computed tomography (SPECT) system, including: a bed configured to support a subject, a source being disposed inside of the subject; an array of detectors configured to detect, at a nonplanar detection surface, primary photons emitted from the source; at least one processor; and memory storing instructions that, when executed by the at least one processor, cause the at least one processor to perform operations including: generating an image of the source based on the primary photons detected by the array of the detectors.

2. The SPECT system of clause 1, wherein the bed includes a material that is transparent to at least a portion of the primary photons.

3. The SPECT system of clause 1 or 2, wherein the array of detectors includes rows of detectors extending in a first direction and columns of detectors extending in a second direction, and wherein the one or more convex portions extend in a third direction that crosses the first direction and the second direction.

4. The SPECT system of one of clauses 1 to 3, wherein the primary photons include gamma rays.

5. The SPECT system of one of clauses 1 to 4, wherein the array of detectors include: scintillator crystals configured to receive the primary photons.

6. The SPECT system of clause 5, wherein the scintillator crystals include an example scintillator crystal, and wherein a detection face of the example scintillator crystal along the detection surface includes one or more convex portions.

7. The SPECT system of clause 5 or 6, wherein the scintillator crystals include an example scintillator crystal, and wherein a detection face of an example scintillator crystal along the detection surface is nonplanar.

8. The SPECT system of one of clauses 5 to 7, wherein the scintillator crystals include an example scintillator crystal, and wherein a detection face of an example scintillator crystal along the detection surface includes one or more ridges.

9. The SPECT system of one of clauses 5 to 8, wherein the scintillator crystals include a first scintillator crystal and a second scintillator crystal, and wherein a detection face of the first scintillator crystal along the detection surface is disposed between the source and a face of the second scintillator crystal along the detection surface, the first scintillator crystal blocking the primary photons from being received by the second scintillator crystal.

10. The SPECT system of one of clauses 5 to 9, wherein the scintillator crystals include an example scintillator crystal, and wherein the example scintillator crystal includes a Fresnel lens.

11. The SPECT system of one of clauses 5 to 10, wherein the scintillator crystals include at least one of cerium-doped multicomponent gadolinium aluminum gallium garnet (Ce: GAGG) or an alloy of cadmium telluride and zinc telluride.

12. The SPECT system of one of clauses 5 to 11, wherein the scintillator crystals are configured to generate secondary photons based on the primary photons, wherein the array of detectors further include sensors coupled to the scintillator crystals and configured to detect the secondary photons, and wherein generating the image of the source is based on the secondary photons detected by the sensors.

13. The SPECT system of clause 12, wherein the array of detectors further includes: barriers disposed between the scintillator crystals, the barriers including a material configured to reflect at least a portion of the secondary photons.

14. The SPECT system of system 12 or 13, wherein an energy of the primary photons is greater than an energy of the secondary photons.

15. The SPECT system of one of clauses 1 to 14, wherein the detection surface includes one or more concave portions.

16. The SPECT system of clause 15, wherein the detector array further includes: an optically transparent material disposed in the one or more concave portions.

17. The SPECT system of one of clauses 1 to 16, wherein the detectors include an example detector, the example detector including a detection face along the detection surface of the array, and wherein the example detector is configured to detect, during a time interval, a number of the primary photons when the detection face is disposed at an angle, the number of the primary photons being based on the angle.

18. The SPECT system of one of clauses 1 to 17, wherein the number of the primary photons detected by the example detector during the time interval is based on the following equation:

$$\frac{y}{\sqrt{{x}^{2} + {y}^{2}}} ,$$

and
wherein y is a distance between the example detector and the source and x is a location of the example detector along the array.

19. The SPECT system of one of clauses 1 to 18, wherein the detectors include an example detector, the example detector including a detection face along the detection surface of the array, and wherein the example detector is configured to detect, during a time interval, a number of the primary photons when the detection face is disposed at a distance from the source, the number of the primary photons being based on the distance.

20. The SPECT system of clause 19, wherein the number of the primary photons detected by the example detector during the time interval is based on the following equation:

$$\frac{1}{\sqrt{{( {x}^{2} + {y}^{2} )}^{2}}} ,$$

and wherein y is a distance between the example detector and the source and x is a location of the example detector along the array.

21. The SPECT system of one of clauses 1 to 20, wherein the array of detectors is noncollimated.

22. The SPECT system of one of clauses 1 to 21, wherein the image includes a pixel or voxel corresponding to a region of a field-of-view (FOV) including the source, wherein the detectors include a first detector and a second detector, and wherein the processor is configured to generate the image by: determining a sensitivity of the first detector to the region of the FOV, one or more lines of response (LORs) extending from the region of the FOV to the first detector, the sensitivity being based on at least one of: at least one of the LORs intersecting the second detector; a distance between the region and the first detector; or one or more angles between a detection face of the first detector along the detection surface and the one or more LORs; and determining a value of the pixel or voxel based on the sensitivity and an amount of the primary photons detected by the first detector.

23. The SPECT system of clause 22, the sensitivity being a first sensitivity, the one or more LORs being one or more first LORs, and wherein the processor is configured to determine the value of the pixel or voxel further based on a sensitivity of the second detector to the region, the sensitivity of the second detector being based on one or more second LORs extending from the region of the FOV to the second detector.

24. The SPECT system of one of clauses 1 to 23, further including: a movement system configured to: move the array of detectors along at least one direction; and rotate the array of detectors along at least one axis.

25. The SPECT system of clause 24, wherein the detectors include an example detector, wherein the actuator is configured to move the example detector between: a first location and first rotation; and a second location and a second rotation, wherein the example detector is configured to detect a first portion of the primary photons when the example detector is disposed at the first location and the first rotation and to detect a second portion of the primary photons when the example detector is disposed at the second location and the second rotation, and wherein the processor is configured to generate the image based on the first portion of the primary photons, the first location, the first rotation, the second portion of the primary photons, the second location, and the second rotation.

26. The SPECT system of clause 25, wherein the processor is configured to generate the image by: determining a first difference between the first portion of the primary photons and the second portion of the primary photons; determining a second difference between a time at which the first portion of the primary photons was detected by the example detector and a time at which the second portion of the primary photons was detected by the example detector; determining a quotient including the first difference divided by the second difference; generating a flux-per-line of response (LOR) distribution based on the quotient; and generating the image by applying weighted least squares, expectation maximization, analytic reconstruction, or maximum likelihood estimation method (MLEM) to the flux-per-LOR distribution.

27. The SPECT system of clause 25 or 26, wherein the example detector has a first sensitivity to a region of a field-of-view (FOV) when the example detector is disposed at the first location and the first rotation, wherein the example detector has a second sensitivity to the region of the FOV when the example detector is disposed at the second location and the second rotation, and wherein the processor is configured to generate the image by: determining a value of a pixel or voxel corresponding to the region of the FOV based on the first sensitivity, the second sensitivity, the first portion of the primary photons, the first location, the first rotation, the second portion of the primary photons, the second location, and the second rotation.

28. The SPECT system of clause 27, wherein at least one additional detector among the detectors is disposed between the region of the FOV and a detection face of the example detector when the example detector is disposed at the first location and the first rotation, and wherein the at least one additional detector is absent between the region of the FOV and the detection face of the example detector when the example detector is disposed at the second location and the second rotation.

29. The SPECT system of clause 27 or 28, wherein a detection face of the example detector is disposed at a first angle with respect to a first line-of-response (LOR) extending from the region to the detection face when the example detector is disposed at the first location and the first rotation, and wherein the detection face of the example detector is disposed at a second angle with respect to a second LOR extending from the region to the detection face when the example detector is disposed at the second location and the second rotation.

30. The SPECT system of one of clauses 27 to 29, wherein a detection face of the example detector is disposed at a first distance from the region when the example detector is disposed at the first location and the first rotation, and wherein the detection face of the example detector is disposed at a second distance from the region when the example detector is disposed at the second location and the second rotation.

31. The SPECT system of one of clauses 1 to 30, wherein generating the image includes: determining a derivative of a flux of the primary photons detected by an example detector among the detectors with respect to time; and generating the image based on the derivative of the flux.

32. The SPECT system of one of clauses 1 to 31, wherein generating the image includes: generating, based on a topography of the detection surface, a systems matrix (P) including sensitivities of the detectors to lines of response (LORs) extending from regions of a field-of-view (FOV), the regions of the FOV respectively corresponding to pixels or voxels of the image, the source being located in the FOV; generating a data array (g) including fluxes of the primary photons detected by the sensors during multiple time intervals; and determining an image array (f) based on the following equation:

$$P f = g ,$$

and
wherein f includes values of the pixels or voxels of the image.

33. The SPECT system of clause 32, wherein the sensitivities of the detectors are based on shadows cast by at least a first portion of the detectors on at least a second portion of the detectors.

34. The SPECT system of clause 32 or 33, wherein the sensitivities of the detectors are based on angles between the LORs and the detection surface.

35. The SPECT system of one of clauses 32 to 34, wherein the sensitivities of the detectors are based on distances between the regions of the FOV and the detectors.

36. The SPECT system of one of clauses 1 to 35, wherein the image is a three-dimensional (3D) image of a field-of-view (FOV) of the SPECT system, the FOV including the source.

37. The SPECT system of one of clauses 1 to 36, wherein the image is indicative of a physiological structure and/or a physiological function of the subject.

38. The SPECT system of one of clauses 1 to 37, further including: a display configured to output the image.

39. The SPECT system of one of clauses 1 to 38, further including: a transceiver configured to transmit data indicative of the image to an external device.

40. A SPECT system, including: a bed configured to support a subject, a source emitting primary photons being disposed inside of the subject; an array of detectors configured to detect a first portion of the primary photons emitted from the source at a first time and to detect a second portion of the primary photons emitted from the source at a second time; a movement system configured to move the array of detectors from a first location and a first rotation at the first time to a second location and a second rotation at the second time; at least one processor; and memory storing instructions that, when executed by the at least one processor, cause the at least one processor to perform operations including: generating an image of the source based on the first portion of the primary photons, the first location, the first rotation, the second portion of the primary photons, the second location, and the second rotation.

41. The SPECT system of clause 40, wherein the movement system is configured to: move the array of detectors along at least one direction; and rotate the array of detectors along at least one axis.

42. The SPECT system of clause 40 or 41, wherein the processor is configured to generate the image by: determining first differences between the first portion of the primary photons and the second portion of the primary photons; determining a second difference between the first time and the second time; determining quotients including the first differences divided by the second difference; generating flux-per-line of response (LOR) distributions based on the quotients; and generating the image based on the flux-per-LOR distributions.

43. The SPECT system of clause 42, wherein the processor is configured to generate the image based on the flux-per-LOR distributions by applying weighted least squares, expectation maximization, analytic reconstruction, or MLEM to the flux-per-LOR distributions.

44. The SPECT system of one of clauses 40 to 43, wherein the detectors have first sensitivities to a region of a field-of-view (FOV) when the array is disposed at the first location and the first rotation, wherein the detectors have second sensitivities to the region of the FOV when the array is disposed at the second location and the second rotation, and wherein the processor is configured to generate the image by: determining a value of a pixel or voxel corresponding to the region of the FOV based on the first sensitivities, the second sensitivities, the first portion of the primary photons, the first location, the first rotation, the second portion of the primary photons, the second location, and the second rotation.

45. The SPECT system of clause 44, wherein detection faces of the detectors are disposed at first angles with respect to a first LOR extending from the region to the detection faces when the array is disposed at the first location and the first rotation, and wherein the detection faces of the detectors are disposed at second angles with respect to a second LOR extending from the region to the detection faces when the example array is disposed at the second location and the second rotation.

46. The SPECT system of clause 44 or 45, wherein detection faces of the detectors are disposed at first distances from the region when the array is disposed at the first location and the first rotation, and wherein the detection faces of the detectors are disposed at second distances from the region when the array is disposed at the second location and the second rotation.

47. The SPECT system of one of clauses 40 to 46, wherein a detection surface of the array of detectors is nonplanar.

48. A SPECT detector, including: a scintillator crystal including a nonplanar detection surface, the scintillator crystal being configured to receive primary photons at the nonplanar detection surface and to generate secondary photons based on the primary photons; and a sensor coupled to the scintillator crystal, the sensor being configured to detect the secondary photons.

49. The SPECT detector of clause 48, wherein the nonplanar detection surface includes one or more concave portions.

50. The SPECT detector of clause 49, further including: an optically transparent material disposed in the one or more concave portions.

51. The SPECT detector of one of clauses 48 to 50, wherein the nonplanar detection surface includes one or more convex portions.

52. The SPECT detector of one of clauses 48 to 51, wherein the nonplanar detection surface includes one or more ridges.

53. The SPECT detector of one of clauses 48 to 52, wherein the scintillator crystal includes cerium-doped multicomponent gadolinium aluminum gallium garnet (Ce: GAGG).

54. The SPECT detector of one of clauses 48 to 53, wherein the sensor includes a photomultiplier.

55. A method, including: identifying a first number of photons detected by a detector during a first time and when the detector is disposed at a first location and/or first rotation; identifying a second number of photons detected by the detector during a second time and when the detector is disposed at a second location and/or second rotation; and determining a value of a pixel or voxel of an image corresponding to a region of a field-of-view (FOV) based on the first number of photons, the first location and/or the first rotation, the second number of photons, and the second location and/or the second rotation.

56. The method of clause 55, wherein a detection face of the detector is nonplanar.

57. The method of clause 55 or 56, wherein the detector is among an array of detectors, and wherein a detection surface of the array of detectors is nonplanar.

58. The method of one of clauses 55 to 57, wherein the value of the pixel or voxel of the image corresponding to the region of the FOV is further based on a topography of the detection surface.

59. The method of one of clauses 55 to 58, further including: identifying a first sensitivity of the detector to one or more lines of response (LORs) extending from the region to the detector positioned at the first location and/or the first rotation; identifying a second sensitivity of the detector to the one or more LORs extending from the region to the detector positioned at the second location and/or the second rotation; and wherein determining the value of the pixel or voxel is based on the first sensitivity and the second sensitivity.

60. The method of clause 59, wherein the first sensitivity is different than the second sensitivity.

61. The method of one of clauses 55 to 60, wherein an additional detector blocks photons emitted from the region from being received by the detector when the detector is positioned at the first location and/or the second rotation or the second location and/or the second rotation.

62. The method of one of clauses 55 to 61, wherein generating the image includes: determining a derivative of a flux of the photons detected by the detector with respect to time; and generating the image based on the derivative of the flux.

63. A method, including: identifying numbers of photons detected by an array of detectors over time, wherein the array of detectors: has a nonplanar detection surface; or is moved over time; and generating an image of the source based on the number of photons detected by the sensors over time.

64. A non-transitory, computer-readable medium storing instructions for performing the method of one of clauses 55 to 63.

65. A system, including: at least one processor; and memory storing instructions that, when executed by the at least one processor, cause the at least one processor to perform operations including the method of one of clauses 55 to 63.

The features disclosed in the foregoing description, or the following claims, or the accompanying drawings, expressed in their specific forms or in terms of a means for performing the disclosed function, or a method or process for attaining the disclosed result, as appropriate, may, separately, or in any combination of such features, be used for realizing implementations of the disclosure in diverse forms thereof.

As will be understood by one of ordinary skill in the art, each implementation disclosed herein can comprise, consist essentially of or consist of its particular stated element, step, or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the implementation to the specified elements, steps, ingredients or components and to those that do not materially affect the implementation. As used herein, the term “based on” is equivalent to “based at least partly on,” unless otherwise specified.

Unless otherwise indicated, all numbers expressing quantities, properties, conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing implementations (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate implementations of the disclosure and does not pose a limitation on the scope of the disclosure. No language in the specification should be construed as indicating any non-claimed element essential to the practice of implementations of the disclosure.

Groupings of alternative elements or implementations disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

1. A single photon emission computed tomography (SPECT) system, comprising:
a bed configured to support a subject, a source being disposed inside of the subject;
an array of detectors configured to detect, at a nonplanar detection surface comprising one or more concave portions, primary photons emitted from the source;
at least one processor; and
memory storing instructions that, when executed by the at least one processor, cause the at least one processor to perform operations comprising:
generating an image of the source based on the primary photons detected by the array of the detectors.

2. (canceled)

3. The SPECT system of claim 1, wherein the array of detectors comprises rows of detectors extending in a first direction and columns of detectors extending in a second direction, and
wherein one or more convex portions extend in a third direction that crosses the first direction and the second direction.

4. (canceled)

5. The SPECT system of claim 1, wherein the array of detectors comprise:
scintillator crystals configured to receive the primary photons.

6. (canceled)

7. The SPECT system of claim 5, wherein the scintillator crystals comprise an example scintillator crystal, and
wherein a detection face of an example scintillator crystal along the detection surface:
is nonplanar;
comprises one or more ridges; or
comprises one or more convex portions.

8. (canceled)

9. The SPECT system of claim 5, wherein the scintillator crystals comprise a first scintillator crystal and a second scintillator crystal, and
wherein a detection face of the first scintillator crystal along the detection surface is disposed between the source and a face of the second scintillator crystal along the detection surface, the first scintillator crystal blocking the primary photons from being received by the second scintillator crystal.

10-15. (canceled)

16. The SPECT system of claim 1, wherein the detector array further comprises:
an optically transparent material disposed in the one or more concave portions.

17. The SPECT system of claim 1, wherein the detectors comprise an example detector, the example detector comprising a detection face along the detection surface of the array, and
wherein the example detector is configured to detect, during a time interval, a number of the primary photons when the detection face is disposed at an angle, the number of the primary photons being based on the angle.

18. The SPECT system of claim 1, wherein the detectors comprise an example detector, the example detector comprising a detection face along the detection surface of the array, and
wherein the example detector is configured to detect, during a time interval, a number of the primary photons when the detection face is disposed at a distance from the source, the number of the primary photons being based on the distance.

19. (canceled)

20. The SPECT system of claim 1, wherein the image comprises a pixel or voxel corresponding to a region of a field-of-view (FOV) comprising the source,
wherein the detectors comprise a first detector and a second detector, and
wherein the processor is configured to generate the image by:
determining a sensitivity of the first detector to the region of the FOV, one or more lines of response (LORs) extending from the region of the FOV to the first detector, the sensitivity being based on at least one of:
at least one of the LORs intersecting the second detector;
a distance between the region and the first detector; or
one or more angles between a detection face of the first detector along the detection surface and the one or more LORs; and
determining a value of the pixel or voxel based on the sensitivity and an amount of the primary photons detected by the first detector.

21. The SPECT system of claim 20, the sensitivity being a first sensitivity, the one or more LORs being one or more first LORs, and
wherein the processor is configured to determine the value of the pixel or voxel further based on a sensitivity of the second detector to the region, the sensitivity of the second detector being based on one or more second LORs extending from the region of the FOV to the second detector.

22. (canceled)

23. The SPECT system of claim 1, further comprising:
a movement system configured to:
move the array of detectors along at least one direction; and
rotate the array of detectors along at least one axis, wherein the detectors comprise an example detector,
wherein the movement system is configured to move the example detector between:
a first location and first rotation; and
a second location and a second rotation,
wherein the example detector is configured to detect a first portion of the primary photons when the example detector is disposed at the first location and the first rotation and to detect a second portion of the primary photons when the example detector is disposed at the second location and the second rotation, and
wherein the processor is configured to generate the image based on the first portion of the primary photons, the first location, the first rotation, the second portion of the primary photons, the second location, and the second rotation.

24. The SPECT system of claim 23, wherein the processor is configured to generate the image by:
determining a first difference between the first portion of the primary photons and the second portion of the primary photons;
determining a second difference between a time at which the first portion of the primary photons was detected by the example detector and a time at which the second portion of the primary photons was detected by the example detector;
determining a quotient comprising the first difference divided by the second difference;
generating a flux-per-line of response (LOR) distribution based on the quotient; and
generating the image by applying weighted least squares, expectation maximization, analytic reconstruction, or maximum likelihood estimation method (MLEM) to the flux-per-LOR distribution.

25. The SPECT system of claim 23, wherein the example detector has a first sensitivity to a region of a field-of-view (FOV) when the example detector is disposed at the first location and the first rotation,
wherein the example detector has a second sensitivity to the region of the FOV when the example detector is disposed at the second location and the second rotation, and
wherein the processor is configured to generate the image by:
determining a value of a pixel or voxel corresponding to the region of the FOV based on the first sensitivity, the second sensitivity, the first portion of the primary photons, the first location, the first rotation, the second portion of the primary photons, the second location, and the second rotation.

26-28. (canceled)

29. The SPECT system of claim 1, wherein generating the image comprises:
determining a derivative of a flux of the primary photons detected by an example detector among the detectors with respect to time; and
generating the image based on the derivative of the flux.

30. The SPECT system of claim 1, wherein generating the image comprises:
generating, based on a topography of the detection surface, a systems matrix (P) comprising sensitivities of the detectors to lines of response (LORs) extending from regions of a field-of-view (FOV), the regions of the FOV respectively corresponding to pixels or voxels of the image, the source being located in the FOV;
generating a data array (g) comprising fluxes of the primary photons detected by the detectors during multiple time intervals; and
determining an image array (f) based on the following equation:
$$P f = g ,$$
and
wherein f comprises values of the pixels or voxels of the image.

31. The SPECT system of claim 30, wherein the sensitivities of the detectors are based on at least one of:
shadows cast by at least a first portion of the detectors on at least a second portion of the detectors;
angles between the LORs and the detection surface; or
distances between the regions of the FOV and the detectors.

32-37. (canceled)

38. A SPECT system, comprising:
a bed configured to support a subject, a source emitting primary photons being disposed inside of the subject;
an array of detectors configured to detect a first portion of the primary photons emitted from the source at a first time and to detect a second portion of the primary photons emitted from the source at a second time;
a movement system configured to move the array of detectors from a first location and a first rotation at the first time to a second location and a second rotation at the second time;
at least one processor; and
memory storing instructions that, when executed by the at least one processor, cause the at least one processor to perform operations comprising:
generating an image of the source based on the first portion of the primary photons, the first location, the first rotation, the second portion of the primary photons, the second location, and the second rotation.

39. (canceled)

40. The SPECT system of claim 38, wherein the processor is configured to generate the image by:
determining first differences between the first portion of the primary photons and the second portion of the primary photons;
determining a second difference between the first time and the second time;
determining quotients comprising the first differences divided by the second difference;
generating flux-per-line of response (LOR) distributions based on the quotients; and
generating the image based on the flux-per-LOR distributions, and
wherein the processor is configured to generate the image based on the flux-per-LOR distributions by applying weighted least squares, expectation maximization, analytic reconstruction, or MLEM to the flux-per-LOR distributions.

41. (canceled)

42. The SPECT system of claim 38, wherein the detectors have first sensitivities to a region of a field-of-view (FOV) when the array is disposed at the first location and the first rotation,
wherein the detectors have second sensitivities to the region of the FOV when the array is disposed at the second location and the second rotation,
wherein the processor is configured to generate the image by:
determining a value of a pixel or voxel corresponding to the region of the FOV based on the first sensitivities, the second sensitivities, the first portion of the primary photons, the first location, the first rotation, the second portion of the primary photons, the second location, and the second rotation.

43-60. (canceled)

61. A method, comprising:
identifying numbers of photons detected by an array of detectors over time, wherein the array of detectors:
has a nonplanar detection surface; or
is moved over time;
determining a derivative of a flux of the numbers of photons detected by the detectors with respect to time; and
generating an image of a source based on the derivative of the flux.

62-63. (canceled)
